SEQUENCE LISTING (10 sequences)

SEQ ID NO: 1 - DNA, 23 bp, synthetic construct
  source 1..23: mol_type = other DNA
  modified_base 1..2: mod_base = OTHER; note = phosphorothioate internucleotide linkages
  modified_base 3..5: mod_base = OTHER; note = phosphorothioate bonds
  modified_base 6..8: mod_base = OTHER; note = phosphorothioate bonds
  modified_base 9..12: mod_base = OTHER; note = phosphorothioate bonds
  modified_base 13..17: mod_base = OTHER; note = phosphorothioate bonds
  modified_base 18..23: mod_base = OTHER; note = phosphorothioate bonds
tcgtcgacgatcggcgcgcgccg

SEQ ID NO: 2 - DNA, 23 bp, synthetic construct
  source 1..23: mol_type = other DNA
  modified_base 1..2: mod_base = OTHER; note = phosphorothioate bonds
  modified_base 3..23: mod_base = OTHER; note = phosphorothioate bonds
tcgacgtcgatcggcgcgcgccg

SEQ ID NO: 3 - DNA, 24 bp, synthetic construct
  source 1..24: mol_type = other DNA
  modified_base 1..24: mod_base = OTHER; note = phosphorothioate bonds
tcgacgtcgatcggcgcgcgccgt

SEQ ID NO: 4 - DNA, 23 bp, synthetic construct
  source 1..23: mol_type = other DNA
  modified_base 1..2: mod_base = OTHER; note = phosphorothioate bonds
  modified_base 3..23: mod_base = OTHER; note = phosphorothioate bonds
  modified_base 1: mod_base = OTHER; note = 5'-Iodo-2'-deoxyuridine
ncgacgtcgatcggcgcgcgccg

SEQ ID NO: 5 - DNA, 24 bp, synthetic construct
  source 1..24: mol_type = other DNA
  modified_base 1..2: mod_base = OTHER; note = phosphorothioate bonds
  modified_base 3..24: mod_base = OTHER; note = phosphorothioate bonds
ncgacgtcgatcggcgcgcgccgt

SEQ ID NO: 6 - DNA, 24 bp, synthetic construct
  source 1..24: mol_type = other DNA
  modified_base 1..24: mod_base = OTHER; note = phosphorothioate bonds
ncgacgtcgatcggcgcgcgccgt

SEQ ID NO: 7 - DNA, 23 bp, synthetic construct
  source 1..23: mol_type = other DNA
  modified_base 1..2: mod_base = OTHER; note = phosphorothioate bonds
  modified_base 3..23: mod_base = OTHER; note = phosphorothioate bonds
ncgacgtcgatcggcgcgcgccg

SEQ ID NO: 8 - DNA, 24 bp, synthetic construct
  source 1..24: mol_type = other DNA
  modified_base 1..2: mod_base = OTHER; note = phosphorothioate bonds
  modified_base 3..24: mod_base = OTHER; note = phosphorothioate bonds
ncgtcgacgatcggcggccgccgt

SEQ ID NO: 9 - DNA, 24 bp, synthetic construct
  source 1..24: mol_type = other DNA
  modified_base 1..24: mod_base = OTHER; note = phosphorothioate bonds
ncgtcgacgatcggcggccgccgt

SEQ ID NO: 10 - DNA, 24 bp, synthetic construct
  source 1..24: mol_type = other DNA
  modified_base 1..2: mod_base = OTHER; note = phosphorothioate bonds
  modified_base 3..5: mod_base = OTHER; note = phosphorothioate bonds
  modified_base 6..8: mod_base = OTHER; note = phosphorothioate bonds
  modified_base 9..12: mod_base = OTHER; note = phosphorothioate bonds
  modified_base 13..17: mod_base = OTHER; note = phosphorothioate bonds
  modified_base 18..24: mod_base = OTHER; note = phosphorothioate bonds
tcgtcgacgatcggcgcgcgccgt

(19) United States
(12) Patent Application Publication
(10) Pub. No.: US 2026/0263583 A1
(43) Pub. Date: Sep. 10, 2026

(54) VACCINE COMPOSITIONS AND METHODS FOR CONTROL OF FOOT AND MOUTH DISEASE

(71) Applicants: INDIAN COUNCIL OF AGRICULTURAL RESEARCH, New Delhi (IN); ZOETIS PHARMACEUTICAL RESEARCH PRIVATE LIMITED, Mumbai (IN)

(72) Inventors: Madhusudan HOSAMANI, Hebbal Bengaluru (IN); B.P. SREENIVASA, Hebbal Bengaluru (IN); R.P. TAMIL SELVAN, Hebbal Bengaluru (IN); P. SARAVANAN, Hebbal Bengaluru (IN); Suresh H BASAGOUDANAVAR, Hebbal Bengaluru (IN); Aniket SANYAL, Anandnagar Bhopal (IN); B.P. MISHRA, Karnal (IN); R VENKATARAMANAN, Ganganagar Bengaluru (IN); Raj Kumar SINGH, Izatnagar Bareilly (IN); Paul DOMINOWSKI, Kalamazoo, MI (US); Suman MAHAN, Kalamazoo, MI (US); Duncan MWANGI, Portage, MI (US); Sharat RAI, Portage, MI (US)

(21) Appl. No.: 19/167,830

(22) PCT Filed: Mar. 14, 2024
(86) PCT No.: PCT/IN2024/050264

§ 371 Date: Sep. 22, 2025

(30) Foreign Application Priority Data
Mar. 21, 2023 (IN) 202311019351

(51) Int. Cl. A61K 39/135; A61K 39/00; A61K 39/385; A61K 39/39; A61P 37/04

(52) U.S. Cl. CPC A61K 39/135; A61K 39/385; A61K 39/39; A61P 37/04; A61K 2039/5252; A61K 2039/55561; A61K 2039/6087

(57) ABSTRACT

This disclosure provides a method of preventing foot lesions caused by FMD virus infection in ruminants through the use of vaccine presented as emulsion containing one or several adjuvants and viral antigens.

### THE NAMES OF THE PARTIES TO A JOINT RESEARCH AGREEMENT

The claimed invention was made as a result of activities undertaken within the scope of a joint research agreement between Zoetis Pharmaceutical Research Ltd and the ICAR-Indian Veterinary Research Institute, Hebbal, Bengaluru, Karnataka 560024, India.

### FIELD OF THE INVENTION

This invention is generally in the field of vaccines against Foot and Mouth Disease (FMD)

### BACKGROUND

Foot-and-mouth disease (FMD) is one of the most severe and contagious diseases affecting farm animals. This disease is endemic in numerous countries in the world, especially in Africa, Asia and South America. In addition, epidemic outbreaks can occur periodically. The presence of this disease in a country may have very severe economic consequences resulting from loss of productivity, loss of weight and milk production in infected herds, and from trade embargoes imposed on these countries. The measures taken against this disease consist of strict application of import restrictions, hygiene controls and quarantine, slaughtering sick animals and vaccination programs using inactivated vaccines, either as a preventive measure at the national or regional level, or periodically when an epidemic outbreak occurs.

FMD is characterized by its short incubation period, its highly contagious nature, the formation of ulcers in the mouth and on the feet and sometimes, the death of young animals. FMD affects a number of animal species, in particular cattle, pigs, sheep and goats. The agent responsible for this disease is a ribonucleic acid (RNA) virus belonging to the Aphthovirus genus of the Picornaviridae family (Cooper et al., 1978, Intervirology 10, 165-180). At present, at least seven types of foot-and-mouth disease virus (FMDV) are known: the European types (A, O and C), the African types (SAT1, SAT2 and SAT3) and an Asiatic type (Asia 1). Numerous sub-types have also been distinguished (Kleid et al., 1981, Science 214, 1125-1129).

FMDV is a naked icosahedral virus of about 25 nm in diameter, containing a single-stranded RNA molecule consisting of about 8500 nucleotides, with a positive polarity. This RNA molecule comprises a single open reading frame (ORF), encoding a single polyprotein containing, inter alia, the capsid precursor also known as protein P1. The protein P1 is myristylated at its amino-terminal end. During the maturation process, the protein P1 is cleaved by the protease 3C into three proteins known as VP0, VP1 and VP3 (or 1AB, 1D and 1C respectively; Belsham G. J., Progress in Biophysics and Molecular Biology, 1993, 60, 241-261). In the virion, the protein VP0 is then cleaved into two proteins, VP4 and VP2 (or 1A and 1B respectively). The mechanism for the conversion of the proteins VP0 into VP1 and VP3, and for the formation of mature virions is not known. The proteins VP1, VP2 and VP3 have a molecular weight of about 26,000 Da, while the protein VP4 is smaller at about 8,000 Da.

Many proposals have been developed in an attempt to design effective vaccines against FMD. Cao et al. (Antiviral Research, 2013, 97:145-153; Veterinary Microbiology, 2014, 168:294-301) reported the design of specific epitope proteins with immunogenicity against FMDV challenge. A synthetic polypeptide corresponding to the fusion of one T epitope and two B epitopes from Asia serotype was proved to induce a protective response (Ren et al., Vaccine, 2011, 29:7960-7965).

Most FMD vaccines require a vaccination regimen entailing two doses given either at 28 days or 6-month interval resulting in poor compliance by farmers and causing some constraint to provide effective control of the disease. There are no FMD vaccines that provide a duration of immunity longer than six months. Office of International Epizootics (OIE) emphasizes the need for a two-dose (prime-boost regimen) and semi-annual revaccinations.

### SUMMARY OF INVENTION

In one aspect, the invention provides a vaccine comprising an FMD antigen and an adjuvant comprising oil, a polycationic carrier, and an immunostimulatory oligonucleotide, wherein said vaccine is a water-in-oil (W/O) emulsion, for use in preventing foot lesions caused by FMD virus infection in a ruminant, wherein said vaccine is administered as a single prime dose and one or more booster doses, wherein the first of said one or more booster doses is administered 7 to about 12 months after the single prime dose. In certain embodiments, said ruminant is FMD-negative.

In certain embodiments, each consequent booster dose is administered 7 to about 12 months after the previous booster dose, e.g., about 8, or about 9, or about 10, or about 11 or about 12 months after the single prime dose.

In certain embodiments, the cationic carrier is DEAE Dextran which can be present in the amount of 25-250 mg per dose.

In certain embodiments, the immunostimulatory oligonucleotide is present in the amount of 25-250 μg per dose, e.g., in the amount of 50-150 μg per dose.

In certain embodiments, the vaccine comprises at least 40% oil v/v of, e.g., at least 45% oil v/v, at least 48% oil v/v, at least 52% oil v/v, or at least 60% oil v/v. The oil may be a non-metabolizable oil such as light mineral oil.

In certain embodiments, the antigen is from an FMD virus which may belong to serotype O, A, C, Asia 1, SAT1, SAT2, or SAT 3. In certain embodiments, the vaccine is monovalent, and in certain other embodiments, the vaccine is multivalent having combination of different serotypes such as FMD virus of serotype A, O, and/or C. In other embodiments, the vaccine comprises the antigen(s) from FMD virus of serotype A, O, and/or Asia 1 or other serotypes such asSAT1, SAT2, and/or SAT3.

In certain embodiments, the FMD antigen is an inactivated FMD virus. In certain embodiments, the inactivated FMD virus is a recombinant FMD virus. In certain embodiments, the recombinant FMD virus lacks functional leader protein and/or contains one or move DIVA markers, e.g. in 3B and/or 3D protein. In some embodiments, the recombinant virus expresses a capsid from a heterologous FMD strain. In some embodiments, the heterologous FMD strain is from serotype O, A, and/or C. In other embodiments, the heterologous FMD strain is from serotype O, A, and/or Asia 1. In yet other embodiments, the heterologous FMD strain is from serotype SAT1, SAT2, and/or SAT3.

In certain embodiments, the FMD virus is present in the amount of at least 4 μg per dose per strain, e.g., at least 8 μg per dose per strain, or about 10 μg per dose per strain.

### DESCRIPTION OF ILLUSTRATIVE EMBODIMENTS

For a better understanding of the invention, the following non-limiting definitions are provided.

“About” or “approximately,” when used in connection with a measurable numerical variable, refers to the indicated value of the variable and to all values of the variable that are within the experimental error of the indicated value (e.g., within the 95% confidence interval for the mean) or within 10 percent of the indicated value, whichever is greater. With regard to time period, the term “about” refers to the indicated value and to a range within 10% of the indicated value (e.g., “about 8 months” includes 8 months as well as 8 months plus or minus 10%), except the upper limit of “about 11 months” is 12 months and the upper limit of “about 12 months” is 12.5 months.

“Adjuvant” means any substance that increases the humoral or cellular immune response to an antigen. Adjuvants are generally used to accomplish two objectives: the controlled release of antigens from the injection site, and the stimulation of the immune system.

“Antibody” refers to an immunoglobulin molecule that can bind to a specific antigen as the result of an immune response to that antigen.

“Antigen” or “immunogen” refers to any substance that is recognized by the animal's immune system and generates an immune response. The term includes killed, inactivated, attenuated, or modified live bacteria, viruses, or parasites. The term “antigen” also includes polynucleotides, polypeptides, recombinant proteins, synthetic peptides, protein extract, cells (including tumor cells), tissues, polysaccharides, or lipids, or fragments thereof, individually or in any combination thereof. The term antigen also includes antibodies, such as anti-idiotype antibodies or fragments thereof, and to synthetic peptide mimotopes that can mimic an antigen or antigenic determinant (epitope).

“Buffer” means a chemical system that prevents change in the concentration of another chemical substance, e.g., proton donor and acceptor systems serve as buffers preventing marked changes in hydrogen ion concentration (pH). A further example of a buffer is a solution containing a mixture of a weak acid and its salt (conjugate base) or a weak base and its salt (conjugate acid).

“Consequent booster dose” refers to the second booster dose, the third booster dose, and so on. “Previous booster dose” is a dose that immediately precedes the “consequent booster dose”. Thus, if a third booster dose is the consequent booster dose, then its preceding booster dose is a second booster dose. Generally speaking, if dose N is a “consequent booster dose”, then dose N−1 is the previous booster dose for dose N.

“Consisting essentially” as applied to the adjuvant formulations refers to formulation which does not contain unrecited additional adjuvanting or immunomodulating agents in the amounts at which said agent exert measurable adjuvanting or immunomodulating effects.

“Dose” refers to a vaccine or immunogenic composition given to a subject in a single administration. A “first dose” or “priming vaccine” or “single prime dose” refers to the dose of such a composition given on Day 0. A “second dose” or an “annual dose” refers to an amount of such composition given subsequent to the first dose at the indicated intervals, which may or may not be the same vaccine or immunogenic composition as the first dose.

The term “emulsifier” is used broadly in the instant disclosure. It includes substances generally accepted as emulsifiers, e.g., different products of TWEEN® or SPAN® product lines (fatty acid esters of polyethoxylated sorbitol and fatty-acid-substituted sorbitan surfactants, respectively), and different solubility enhancers such as PEG-40 Castor Oil or another PEGylated hydrogenated oil.

The term “FMD-negative” refers to an animal that has not been previously vaccinated against FMD. Preferably, the term “FMD-negative” refers to the animal that has been neither previously vaccinated against FMD nor previously (or currently) infected with FMD.

“Humoral immune response” refers to one that is mediated by antibodies.

“Immune response” in a subject refers to the development of a humoral immune response, a cellular immune response, or a humoral and a cellular immune response to an antigen. Immune responses can usually be determined using standard immunoassays and neutralization assays, which are known in the art.

“Immunologically effective amount” or “effective amount to produce an immune response” of an antigen is an amount effective to induce an immunogenic response in the recipient. The immunogenic response may be sufficient for diagnostic purposes or other testing, or may be adequate to prevent signs or symptoms of disease, including adverse health effects or complications thereof, caused by infection with a disease agent. Either humoral immunity or cell-mediated immunity or both may be induced. The immunogenic response of an animal to an immunogenic composition may be evaluated, e.g., indirectly through measurement of antibody titers, lymphocyte proliferation assays, or directly through monitoring signs and symptoms after challenge with wild type strain, whereas the protective immunity conferred by a vaccine can be evaluated by measuring, e.g., reduction in clinical signs such as mortality, morbidity, temperature number, overall physical condition, and overall health and performance of the subject. The immune response may comprise, without limitation, induction of cellular and/or humoral immunity.

“Immunogenic” means evoking an immune or antigenic response. Thus an immunogenic composition would be any composition that induces an immune response.

“Pharmaceutically acceptable” refers to substances, which are within the scope of sound medical judgment, suitable for use in contact with the tissues of subjects without undue toxicity, irritation, allergic response, and the like, commensurate with a reasonable benefit-to-risk ratio, and effective for their intended use.

The inventors discovered that the vaccines according to any embodiments described above can advantageously provide protection from FMD-caused foot lesions for about twelve months when administered even as a single dose vaccine.

Therefore, the invention provides a vaccine for prevention of FMD-caused foot lesions in an animal, wherein the vaccine is administered in a prime single dose and in one or more booster doses, wherein the first booster dose is administered 7 to about 12 months after the single prime dose, wherein the vaccine comprises an FMD antigen and an adjuvant comprising (or consisting or consisting essentially) an oil, an immunostimulatory oligonucleotide containing CpG, a polycationic carrier and optionally one or more emulsifiers wherein the vaccine is a water-in-oil emulsion. In certain embodiment, the animal is a ruminant, such as a bovine. In certain embodiment, the animal is FMD-negative.

If more than one booster dose is administered, then each consequent booster dose is administered 7 to about 12 months after the previous booster dose. Thus, the first of said one or more booster doses is administered 7 to about 12 months after the prime single dose, the second of said one or more booster doses is administered 7 to about 12 months after the first of said one or more booster doses, the third of said one or more booster doses is administered 7 to about 12 months after the second of said one or more booster doses and so on. In certain embodiments, the interval between the prime single dose and the first booster dose is 7 months, or about 8 months or about 9 months, or about 10 months, or about 11 months or about 12 months.

The intervals between the two doses are selected independently from each other and are, as noted above, between 7 and about 12 months and may be the same or different.

Table 1 provides a non-limiting illustration related to the intervals between the prime single (PS) dose and the first booster dose (B1) and between the first booster dose and the second booster dose (B2). The mark “X” indicates that the proposed regimen is suitable for practicing the invention.

TABLE 1

| Interval | | | | | | |
|---|---|---|---|---|---|---|
| between | | | | | | |
| SP and B1, | Interval between B1 and B2, months | | | | | |
| months | 7 | ~8 | ~9 | ~10 | ~11 | ~12 |
| 7 | X | X | X | X | X | X |
| ~8 | X | X | X | X | X | X |
| ~9 | X | X | X | X | X | X |
| ~10 | X | X | X | X | X | X |
| ~11 | X | X | X | X | X | X |
| ~12 | X | X | X | X | X | X |

Similarly, the time interval between the third booster dose and the second booster dose that is between 7 months and about 12 months is suitable regardless of the specific interval between SP and B1 and between B1 and B2 as long as both of these intervals are between 7 and about 12 months.

The vaccines described herein are suitable for any of the administration regimens disclosed above. The vaccines comprise an FMD antigen and an adjuvant comprising an oil, a polycationic carrier, an immunostimulatory oligonucleotide containing CpG and, optionally, one or more emulsifiers, wherein said vaccine is a W/O emulsion.

### Antigens

Viruses from any FMD serotype(s) are suitable for use with the vaccine of the invention. Currently, seven serotypes of FMD have been isolated. Of the seven serotypes of this virus, A, C, O, Asia 1, and SAT3 appear to be distinct lineages; SAT 1 and SAT 2 are unresolved clades. Within each serotype, multiple strains exist. For example, A24 Cruzeiro belongs to serotype A, and 01 Campos belongs to serotype O.

It should be noted that the antigens of different serotypes may provide limited cross-protection against challenge with a virus of heterologous serotype. U.S. Pat. No. 10,010,605 disclosed the results of vaccination of pigs with VLPs containing peptide pools from FMD viruses of different strains. The authors reported that that specific IFN-gamma response (cellular response) was detected in both Asia1 Shamir VLP groups when Asia1 Shamir FMDV antigens were used, and specific IFN-gamma response (cellular response) was detected in both A22 Iraq VLP groups when A22 Iraq FMDV antigens were used. The Asial Shamir VLP group showed cross-immunogenicity when A22 Iraq FMDV antigens were used. Specific plasma cells (humoral response) were detected in both Asia1 Shamir VLP groups and both A22 Iraq VLP groups with Asia1 Shamir antigens and A22 Iraq antigens, respectively. Cross-immunogenicity (plasma cells) in Asia1 Shamir VLP groups was observed with A22 Iraq antigens. Specific memory B cells (humoral response) were detected in Asia1 Shamir VLP groups and A22 Iraq VLP groups with Asia1 Shamir antigens and A22 Iraq antigens, respectively. Good cross-immunogenicity (B cells) in Asia1 Shamir VLP groups was observed with A22 Iraq antigens. Some cross-immunogenicity (B cells) in A22 Iraq VLP groups was also observed with Asia1 Shamir antigens. The authors concluded VLPs could elicit an immune response sufficient to protect against heterologous challenge.

However, it is preferred that the vaccine contains the antigens from the serotype against which the protection is desirable, which may vary based on the geographic region. Thus, for African region, where SAT-1, SAT-2 and SAT-3 are most prevalent, it is preferred that the vaccine contains the antigens from the strain(s) that belong to serotypes SAT-1, SAT-2 and/or SAT-3. For Europe, where strains of serotypes A, O, and C are prevalent, it is preferred that the vaccine contains antigens from strain(s) that belong to serotype(s) O, A, and C. For Asia, it is preferred that the vaccine contains the antigens from strain(s) that belong to serotype Asia.

FMD virus of any serotype may be used as needed, provided that such virus is not pathogenic. Pathogenicity may be reduced by inactivation of the virus, e.g., treatment with formaldehyde or BEI.

In certain embodiments, the virus may be attenuated by culture passage or via recombinant means. It has previously been demonstrated, for example, that deletion of the leader protein L^{pro} coding region results in FMD virus which is attenuated in cattle and pigs. See, e.g., U.S. Pat. Nos. 5,824,316, 8,765,141, Virology 1997 227(1): 96-102, J. Virol 2012 86:11675-11685. Point mutations in at positions 55 and 58 within the SAP domain of L protein also resulted in a viable virus that displayed a mild attenuated phenotype in cell culture and was protective in swine FMD model. See U.S. Pat. No. 8,846,057.

In certain embodiments, the virus also contains negative antigenic markers which allow for DIVA (differentiating infected from vaccinated animals) assays. In certain embodiments, the negative antigenic markers are introduced to 3D and/or 3B proteins.

Like other viruses, the FMD virus continually evolves and mutates, thus one of the difficulties in vaccinating against it is the huge variation between, and even within, serotypes. There is no cross-protection between serotypes (a vaccine for one serotype will not necessarily protect against any others) and in addition, two strains within a given serotype may have nucleotide sequences that differ by as much as 30% for a given gene. This means FMD vaccines must be specific to the strain involved, although certain cross-protection between strains has been reported.

Thus, in certain embodiments, endonuclease restriction sites are introduced into the genome of the virus, thereby allowing introduction of proteins (e.g., proteins forming the outer capsids) from heterologous FMD strains.

In certain embodiments, the antigen component comprises FMD strain A24 Cruzeiro, which may optionally be modified inactivation of the leader protein (either by substitutions or insertions, or deletions, or combinations thereof), negative marker mutations in 3B and/or 3D proteins, and by introduction of restriction endonuclease sites for an easier introduction of sequences for antigens (e.g., capsid proteins) from heterologous strains. Suitable non-limiting examples of the antigens are described in U.S. Pat. No. 8,765,141. See also U.S. Pat. Nos. 9,180,179 and 10,478,487.

Thus, a DNA sequence complementary to the reference DNA sequence is a template for, i.e. is complementary to or “encodes”, the RNA genome of the FMDV virus (i.e., RNA that encodes the FMDV). In certain embodiments, the virus comprises capsid protein(s) of heterologous FMD strains (i.e., strains of FMD other than A24 Cruzeiro, including without limitations, strains of lineages C, O, Asia 1, SAT3, SAT 1 and SAT 2, Turkey 06 and other strains of lineage A).

In certain non-limiting embodiments, the FMD may be of the following strains O1 Manisa, O1 BFS or Campos, A24 Cruzeiro, Asia 1 Shamir, A Iran '96, A22 Iraq, SAT2 Saudi Arabia strains.

Still other strains may include FMDV strains A10-61, A5, A12, A24/Cruzeiro, C3/Indaial, O1, C1-Santa Pau, C1-C5, A22/550/Azerbaijan/65, SAT1-SAT3, A, A/TNC/71/94, A/IND/2/68, A/IND/3/77, A/IND/5/68, A/IND/7/82, A/IND/16/82, A/IND/17/77, A/IND/17/82, A/IND/19/76, A/IND/20/82, A/IND/22/82, A/IND/25/81, A/IND/26/82, A/IND/54/79, A/IND/57/79, A/IND/73/79, A/IND/85/79, A/IND/86/79, A/APA/25/84, A/APN/41/84, A/APS/44/05, A/APS/50/05, A/APS/55/05, A/APS/66/05, A/APS/68/05, A/BIM/46/95, A/GUM/33/84, A/ORS/66/84, A/ORS/75/88, A/TNAn/60/947/Asia/1, A/IRN/05, Asia/IRN/05, 0/HK/2001, 0/UKG/3952/2001, O/UKG/4141/2001, Asia 1/HNK/CHA/05 (GenBank accession number EF149010, herein incorporated by reference), Asia I/XJ (Li, ZhiYong et al. Chin Sci Bull, 2007), HK/70 (Chin Sci Bull, 2006, 51 (17): 2072-2078), O/UKG/7039/2001, O/UKG/9161/2001, O/UKG/7299/2001, O/UKG/4014/2001, O/UKG/4998/2001, O/UKG/9443/2001, O/UKG/5470/2001, O/UKG/5681/2001, O/ES/2001, HKN/2002, 05India, O/BKF/2/92, K/37/84/A, KEN/1/76/A, GAM/51/98/A, A10/Holland, O/KEN/1/91, O/IND49/97, O/IND65/98, O/IND64/98, O/IND48/98, O/IND47/98, O/IND82/97, O/IND81/99, O/IND81/98, O/IND79/97, O/IND78/97, O/IND75/97, O/IND74/97, O/IND70/97, O/IND66/98, O/IND63/97, O/IND61/97, O/IND57/98, O/IND56/98, O/IND55/98, O/IND54/98, O/IND469/98, O/IND465/97, O/IND464/97, O/IND424/97, O/IND423/97, O/IND420/97, O/IND414/97, O/IND411/97, O/IND410/97, O/IND409/97, O/IND407/97, O/IND399/97, O/IND39/97, O/IND391/97, O/IND38/97, O/IND384/97, O/IND380/97, O/IND37/97, O/IND352/97, O/IND33/97, O/IND31/97, O/IND296/97, O/IND23/99, O/IND463/97, O/IND461/97, O/IND427/98, O/IND28/97, O/IND287/99, O/IND285/99, O/IND282/99, O/IND281/97, O/IND27/97, O/IND278/97, O/IND256/99, O/IND249/99, O/IND210/99, O/IND208/99, O/IND207/99, O/IND205/99, O/IND185/99, O/IND175/99, O/IND170/97, O/IND164/99, O/IND160/99, O/IND153/99, O/IND148/99, O/IND146/99, O/SKR/2000, A22/India/17/77.

Variants of such antigens are also envisioned. The variants are at least 80% identical (e.g., 85% identical, 90% identical, 95% identical, 96% identical, 97% identical, 98% identical or 99% identical) to a reference sequence using one of the alignment programs described using standard parameters. Multiple alignment tools are available to determine sequence identity, including, without limitations, BLAST, CLUSTAL or PHILIP.

One of skill in the art will recognize that these values can be appropriately adjusted to determine corresponding identity of proteins encoded by two nucleotide sequences by taking into account codon degeneracy, amino acid similarity, reading frame positioning, and the like.

In certain embodiments, the variants encompass more than the specific exemplary nucleotide or amino acid sequences and include functional equivalents thereof. Alterations in a nucleic acid fragment that result in the production of a chemically equivalent amino acid at a given site, but do not affect the functional properties of the encoded polypeptide, are well known in the art. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

The polypeptides of the invention may also be altered in various ways including amino acid substitutions, deletions, truncations, and insertions. Novel proteins having properties of interest may be created by combining elements and fragments of proteins of the present invention, as well as with other proteins. Methods for such manipulations are generally known in the art. Thus, the genes and nucleotide sequences of the invention include both the naturally occurring sequences as well as mutant forms. Likewise, the proteins of the invention encompass naturally occurring proteins as well as variations and modified forms thereof. Such variants will continue to possess the desired modified activities of the parent FMD virus. The mutations that will be made in the DNA encoding the variant must not place the sequence out of reading frame and preferably will not create complementary regions that could produce secondary mRNA structure.

Methods of growing and purifying the antigens suitable for the instant invention are well known in the art and include, without limitations, hollow fiber filtration and PEG precipitation. These methods yield somewhat different antigenic compositions. For example, in PEG precipitation, the antigenic composition is depleted of non-structural proteins. In other methods, such as, for example, hollow fiber filtration, the antigenic composition contains both structural and non-structural FMD proteins. Accordingly, in some embodiments, the FMD antigen comprises structural proteins. In other embodiments, such as, for example, where the FMD antigen is prepared by hollow fiber filtration, the FMD antigen comprises both structural and non-structural proteins, particularly 3D protein.

In other embodiments, the antigen may include empty FMD virus capsids or virus-like particles, or VLPs. Both are known in the art. For example, US Published Patent Application 2004/0001864 teaches a vaccine against foot-and-mouth disease wherein empty capsids are produced by coexpressing P1 and protease 3C. U.S. Pat. No. 8,409,588 discloses a method of producing a foot and mouth disease virus-like particle comprising: providing a host cell including an expression system comprising a promoter operably linked to a nucleic acid molecule encoding a poly-protein, said polyprotein comprising FMDV 1A protein-nonFMDV protease recognition sequence-FMDV 1B protein-nonFMDV protease recognition sequence-FMDV 1C protein-nonFMDV protease recognition sequence-FMDV 1D protein, said host cell expressing a protease recognizing said nonFMDV protease recognition sequence; growing the host cell under conditions such that the poly-protein is produced, resolved into 1A, 1B, 1C and 1D by the protease and 1A, 1B, 1C and 1D assemble into virus-like particles; and recovering the virus-like particles. See also Subramanian et al. Development of foot-and-mouth disease virus (FMDV) serotype O virus-like-particles (VLPs) vaccine and evaluation of its potency. Antiviral Res. December 2012; 96(3): 288-95 reporting VLPs resulting from expressing the FMDV structural proteins along with the 3C protease in Sf9 cells.

The vaccines described herein may be monovalent (FMD antigen of only one strain is used) or multivalent (FMD antigens from two or more strains are used). Thus, for example, the vaccine may use the antigen from a single strain of serotype A, or from a single strain of serotype C or from a single strain of serotype C or from a single strain of serotype Asia, and so on. Multivalent vaccines may contain a strain from a serotype A, a strain from serotype O, a strain from serotype C, a strain from serotype Asia, or any two or any three of these. In other embodiments, the multivalent vaccine may contain several strains from the same serotype.

The vaccines described herein allow for certain flexibility in the amount of the antigen per dose. In certain embodiments, the amount of the antigen comprises at least 4 μg of the antigen per dose per strain (e.g., at least 5, at least 6, at least 7, at least 8, at least 9, or at least 10 μg or more per dose per strain). In multivalent vaccines described herein, the amounts of different antigens per dose are selected independently from other antigens.

Additionally, the use of heterologous (non-FMD) viral vectors encoding FMD antigens has been reported to provide protection against a challenge. See, e.g., U.S. Pat. No. 10,188,721 disclosing compositions or vaccines comprising recombinant viral vectors such as adenoviral vectors expressing FMDV antigens that elicit protective response in animals.

Thus, in certain embodiments, the FMD antigen is provided in a form of an adenovirus vector that expresses FMDV structural protein P1 (VP4-VP2-Vp3-VP1), nonstructural protein P2 (2A, 2B, and 2C) or nonstructural protein P3 (3A, 3B, 3C and 3D) or active fragment or variant thereof.

In other embodiments, the antigen may comprise at least one immunogenic peptide from the FMD virus. Suitable non-limiting examples of such peptides are disclosed, for example, in WO03068169, 20110206718, and Ren et al., Vaccine 29 (2011) 7960-7965, incorporated herein by reference.

Once the immunogenicity of a given antigen is determined relative to inactivated virus by standard methods, one of ordinary skill in the art would have no difficulties in selecting the appropriate dose of the antigen. In multivalent vaccines described herein, the amounts of different antigens per dose are selected independently from other antigens.

### Adjuvants

The vaccine according to the invention is formulated as a water-in-oil emulsion (W/O emulsion) and comprises (or consists essentially of or consists) an oil, an immunostimulatory oligonucleotide comprising CpG, a polycationic carrier and, optionally, one or more emulsifiers.

Different oils are suitable for the invention including non-metabolizable oils (e.g. a light mineral oil), metabolizable oils (e.g., e.g., vegetable oil, or fatty acid, polyol or alcohol esters) or a mixture thereof. If a mixture of non-metabolizable oils and metabolizable oils is used, it is preferred that the non-metabolizable oils comprise over 50% v/v (e.g, over 60%, or over 70% or over 80% or over 90% or over 95% or over 99%) of the mixture. In certain embodiments, the 100% of the oil in the emulsion is non-metabolizable. In the most preferred embodiments, the 100% of the oil in the W/O emulsion is a mineral oil. As used herein, the term “mineral oil” refers to a mixture of liquid hydrocarbons obtained from petrolatum via a distillation technique. The term is synonymous with “liquefied paraffin”, “liquid petrolatum” and “white mineral oil.” The term is also intended to include “light mineral oil,” i.e., oil which is similarly obtained by distillation of petrolatum, but which has a slightly lower specific gravity than white mineral oil. See, e.g., Remington's Pharmaceutical Sciences, 18th Edition (Easton, Pa.: Mack Publishing Company, 1990, at pages 788 and 1323). Mineral oil can be obtained from various commercial sources, for example, J. T. Baker (Phillipsburg, Pa.) or USB Corporation (Cleveland, Ohio). Preferred mineral oil is light mineral oil commercially available under the name DRAKEOL®.

The volume fraction of the oil in the vaccine should be sufficient for the W/O emulsion. Water-in-oil emulsion's integrity can be maintained as long as the dispersed spherical water droplets are not present in a more concentrated form than the maximum packing fraction for random packing of monodisperse droplets, i.e.: 0.64. See Tadros, Emulsion Formation, Stability and Rheology, 1st ed. 2013, Wiley-VCH GmbH & Co KGaA. As long as the total volume fraction occupied by the aqueous droplets does not exceed 0.64, i.e.: 64% v/v. Thus, in different embodiments, the oil can be present in the amount of greater than 36% of the vaccine v/v (e.g, 37%, 38%, 39%, 40%, 41%, 42%, 43%, 44%, 45%, 46%, 47%, 48%, 49%, 50%, 51%, 52%, 53%, 55%, 60%, 65%, 70%, 75%, 80% or more of the vaccine v/v).

The emulsion may also comprise one or more emulsifiers. In additional embodiments, the emulsifiers used herein do not include lecithin, or use lecithin in an amount which is not immunologically effective.

Non-natural, synthetic emulsifiers suitable for use in the adjuvant formulations of the present invention include sorbitan-based non-ionic surfactants, e.g. fatty-acid-substituted sorbitan surfactants (commercially available under the name SPAN® or ARLACEL®), fatty acid esters of polyethoxylated sorbitol (TWEEN®), polyethylene glycol esters of fatty acids from sources such as castor oil (EMULFOR®); polyethoxylated fatty acid (e.g., stearic acid available under the name SIMULSOLOM-53), polyethoxylated isooctylphenol/formaldehyde polymer (TYLOXAPOL®), polyoxyethylene fatty alcohol ethers (BRIJ®); polyoxyethylene nonphenyl ethers (TRITON® N), polyoxyethylene isooctylphenyl ethers (TRITON® X). Preferred synthetic surfactants are the surfactants available under the name SPAN® and TWEEN®, such as TWEEN®-80 (Polyoxyethylene (20) sorbitan monooleate) and SPAN®-80 (sorbitan monooleate).

The emulsifiers generally may be present in the emulsion in the amounts of 0 to 20% v/v. In certain embodiments, TWEEN® is present in the amount of 1% to 10% v/v of the vaccine and SPAN® is also present in the amount of 1% to 10% v/v of the vaccine. Thus, for example, TWEEN® is present in the amount of 1% to 6% v/v of the vaccine and SPAN® is also present in the amount of 1% to 6% v/v of the vaccine. In other embodiments, TWEEN® is present in the amount of 1% to 2% v/v of the vaccine and SPAN® is present in the amount of 5% to 8% v/v of the vaccine. In yet other embodiments, TWEEN® is present in the amount of 4% to 7% v/v of the vaccine and SPAN® is present in the amount of 1% to 3% v/v of the vaccine.

The adjuvant component of the vaccine further comprises a polycationic carrier. Suitable non-limiting examples of polycationic carriers include, without limitations, PEGs, guar gums, chitosan derivatives, polycellulose derivatives like hydroxyethyl cellulose (HEC) polyethylenimene, poly aminos like polylysine, cationized dextran and the like. In certain embodiments, the polycationic carrier is Diethylaminoethyl (DEAE) Dextran.

The polycationic carrier, such as DEAE Dextran, may be present in the vaccine in the amount of between 5 and about 500 mg per dose (e.g., 10-500 mg, or 10-300 mg, or 10-50 mg, or 50-200 mg or about 100 mg per dose).

The adjuvant component of the vaccine also comprises an immunostimulatory oligonucleotide. Immunomodulatory oligonucleotides according to the invention comprise CpG (and are also referred to as “CpG containing immunostimulatory oligonucleotides”, “CpG oligonucleotides” or simply “CpGs”). The effect of CpG containing oligonucleotides on the immune system has been known for over 20 years.

Generally, the CpGs suitable for the invention are between 15 and 100 bases long, e.g., between 15 and 50 bases long, or between 18 and 40 bases long or between 20 and 30 bases long, or 20-24 bases long.

Several classes of CpG have been described, including A-class CpGs, B-class CpGs, C-class CpGs, and P-class CpGs. In certain embodiments, the CpG containing immunostimulatory oligonucleotide is a P-class CpG. P-class CpGs are characterized by the presence of one or more TLR-9 activating motif(s) and two palindromes or two complementarity areas. Preferably, the one or more TLR-9 activating motifs are at the 5′ of the oligonucleotide and may be completely or partially be incorporated into the 5′ palindrome or the 5′ complementarity area. TLR-9 activating motifs are known and include, without limitations, TCG, TTCG, TTTCG, TYpR, TTYpR, TTTYpR, UCG, UUCG, UUUCG, TTT, or TTTT. The 5′ palindrome or the 5′ complementary area is at least 6 bases long. The 3′ palindrome or the 3′ complementary area is at least 8 bases long and is generally rich in C and G. These structural features of the P-class CpGs confer the ability to spontaneously self-assemble into concatamers either in vitro and/or in vivo.

In order to increase lipophilicity of the CpG oligonucleotides, at least one lipophilic substituted nucleotide analog may be included, preferably at the 5′ end of the oligonucleotide. The P-class immunostimulatory oligonucleotides may be modified according to techniques known in the art. For example, J-modification refers to iodo-modified nucleotides. E-modification refers to ethyl-modified nucleotide(s). Thus, E-modified P-class immunostimulatory oligonucleotides are P-class immunostimulatory oligonucleotides, wherein at least one nucleotide (preferably 5′ nucleotide) is ethylated. Additional modifications include attachment of 6-nitro-benzimidazol, O-Methylation, modification with proynyl-dU, inosine modification, 2-bromovinyl attachment (preferably to uridine).

The oligonucleotides modified by an addition of a lipophilic moiety are generally described in US20100166780.

In certain embodiments, CpGs according to the invention comprise the modified backbone including, without limitations, phosphorothioate modifications, halogenations, alkylation (e.g., ethyl- or methyl-modifications), and phosphodiester modifications.

Suitable non-limiting examples of modified P-class immunostiumulatory oligonucleotides are provided below (“*” refers to a phosphorothioate bond, “-” refers to a phosphodiester bond, “JU” refers to 5′-lodo-2′-deoxyuridine and “EU” refers to 5-Ethyl-2′-deoxyuridine).

SEQ ID NO: 1
5′ T*C-G*T*C-G*A*C-G*A*T*C-G*G*C*G*C-G*C*
G*C*C*G 3′
SEQ ID NO: 2
5′ T*C-G*A*C*G*T*C*G*A*T*C*G*G*C*G*C*G*C*G*C*C*
G 3′
SEQ ID NO: 3
5′ T*C*G*A*C*G*T*C*G*A*T*C*G*G*C*G*C*G*C*G*C*C*
G*T 3′
SEQ ID NO: 4
5′ JU*C-G*A*C*G*T*C*G*A*T*C*G*G*C*G*C*G*C*G*C*C
*G 3′
SEQ ID NO: 5
5′ JU*C-G*A*C*G*T*C*G*A*T*C*G*G*C*G*C*G*C*G*C*
C* G*T 3′
SEQ ID NO: 6
5′ JU*C*G*A*C*G*T*C*G*A*T*C*G*G*C*G*C*G*C*G*C*
C* G*T 3′
SEQ ID NO: 7
5′ EU*C-G*A*C*G*T*C*G*A*T*C*G*G*C*G*C*G*C*G*C*
C*G 3′
SEQ ID NO: 8
5′ JU*C-G*T*C*G*A*C*G*A*T*C*G*G*C*G*G*C*C*G*C*
C* G*T 3′
SEQ ID NO: 9
5′ JU*C*G*T*C*G*A*C*G*A*T*C*G*G*C*G*G*C*C*G*C*
C* G*T 3′
SEQ ID NO: 10
5′ T*C-G*T*C-G*A*C-G*A*T*C-G*G*C*G*C-G*C*G*C*C*
G 3′

In certain embodiments, the CpG oligonucleotide according to the invention comprises any one of SEQ ID NOs 1-10 or an oligonucleotide comprising at least 15 consecutive bases of any one of SEQ ID NOs 1-10. In the most preferred embodiment, the vaccine comprises an oligonucleotide comprising at least 15 consecutive bases of SEQ ID NO: 8 (e.g., at least 16, at least 17, at least 18, at least 19, at least 20, at least 21, at least 22, or at least 23).

The CpG oligonucleotide may be present in the vaccine in the amount of 10-400 μg per dose of the vaccine, or 25-300 or 50-200 or 50-100 mg per dose.

Other components of the compositions can include pharmaceutically acceptable excipients, such as carriers, solvents, and diluents, isotonic agents, buffering agents, stabilizers, preservatives, antibacterial agents, antifungal agents, and the like. Typical carriers, solvents, and diluents include water, saline, dextrose, ethanol, glycerol, oil, and the like. Representative isotonic agents include sodium chloride, dextrose, mannitol, sorbitol, lactose, and the like. Useful stabilizers include gelatin, albumin, and the like. The compositions can also contain antibiotics or preservatives, including, for example, gentamicin, merthiolate, or chlorocresol. The various classes of antibiotics or preservatives from which to select are well known to the skilled artisan.

In certain embodiments, the vaccines of the invention prepared by making a first solution containing the oil and the optional oil-soluble emulsifier, and a second solution, comprising the aqueous diluent, the antigen, the CpG oligonucleotide, the polycationic carrier and the optional water-soluble emulsifier. The first solution is then combined with the second solution, for example, by adding the second solution dropwise into the first solution, and thereby preparing the W/O emulsion vaccine according to the invention.

The disclosure further provides the following items:
Item 1. A vaccine comprising an FMD antigen and an adjuvant comprising (or consisting of or consisting essentially of) oil, a polycationic carrier, and an immunostimulatory oligonucleotide, wherein said vaccine is a water-in-oil (W/O) emulsion, for use in preventing foot lesions caused by FMD infection in a ruminant, wherein said vaccine is administered as a single prime dose and one or more booster doses, wherein the first of said one or more booster doses is administered 7 to about 12 months after the single prime dose.
Item 2. The vaccine according to item 1, wherein each consequent booster dose is administered 7 to about 12 months after the previous booster dose.
Item 3. The vaccine according to item 1 or 2, wherein said first booster dose is administered about 8, or about 9, or about 10, or about 11 or about 12 months after the single prime dose.
Item 4. The vaccine according to any one of item 1-3 wherein the cationic carrier is DEAE Dextran.
Item 5. The vaccine according to item 4, wherein the DEAE Dextran is present in the amount of 25-250 mg per dose.
Item 6. The vaccine according to item 5, wherein the immunostimulatory oligonucleotide is present in the amount of at least 25 μg per dose.
Item 7. The vaccine according to item 6, wherein the immunostimulatory oligonucleotide is present in the amount of 50-150 μg per dose.
Item 8. The vaccine according to any one of items 1-6, wherein the immunostimulatory oligonucleotide is a P-class immunostimulatory oligonucleotide.
Item 9. The vaccine according to item 8, wherein the P-class immunostimulatory oligonucleotide is a modified P-class immunostimulatory oligonucleotide.
Item 10. The vaccine according to item 9, wherein the modified P-class immunostimulatory oligonucleotide comprises 5′-lodo-2′-deoxyuridine or 5-Ethyl-2′-deoxyuridine.
Item 11. The vaccine according to item 10, wherein the P-class immunostimulatory oligonucleotide comprises 15 consecutive oligonucleotides of SEQ ID NO: 8.
Item 12. The vaccine according to item 11, wherein the P-class immunostimulatory oligonucleotide comprises 20 consecutive oligonucleotides of SEQ ID NO: 8.
Item 13. The vaccine according to item 12, wherein the P-class immunostimulatory oligonucleotide comprises SEQ ID NO: 8.
Item 14. The vaccine according to any one of items 1-13 wherein the vaccine comprises at least 40% oil v/v.
Item 15. The vaccine according to item 14 wherein the vaccine comprises at least 45% oil v/v.
Item 16. The vaccine according to item 15 wherein the vaccine comprises at least 48% oil v/v.
Item 17. The vaccine according to item 16, wherein the vaccine comprises at least 52% oil v/v.
Item 18. The vaccine according to item 17, wherein the vaccine comprises at least 60% oil v/v.
Item 19. The vaccine according to any one of items 1-18 wherein the oil is a non-metabilolizable oil.
Item 20. The vaccine according to item 19, wherein the non-metabolizable oil is a light mineral oil.
Item 21. The vaccine according to any one of items 1-20 wherein the FMD antigen is from an FMD virus of Type O, A and/or Asia-1.
Item 22. The vaccine according to any one of items 1-20 wherein the FMD antigen is from an FMD virus of Type O, A and/or C serotype.
Item 23. The vaccine according to any one of items 1-20 wherein the FMD antigen is from an FMD virus of SAT-1, SAT-2 and/or SAT-3 serotypes.
Item 24. The vaccine according to item 24, wherein the FMD antigen is from a strain selected from the group consisting of O1 Manisa, O1 BFS or Campos, Asia 1 Shamir, A Iran '96, A22 Iraq, SAT2 Saudi Arabia, A10-61, A5, A12, A24/Cruzeiro, C3/Indaial, O1, C1-Santa Pau, C1-C5, A22/550/Azerbaijan/65, SAT1-SAT3, A, A/TNC/71/94, A/IND/2/68, A/IND/3/77, A/IND/5/68, A/IND/7/82, A/IND/16/82, A/IND/17/77, A/IND/17/82, A/IND/19/76, A/IND/20/82, A/IND/22/82, A/IND/25/81, A/IND/26/82, A/IND/27/11, A/IND/54/79, A/IND/57/79, A/IND/73/79, A/IND/85/79, A/IND/86/79, A/APA/25/84, A/APN/41/84, A/APS/44/05, A/APS/50/05, A/APS/55/05, A/APS/66/05, A/APS/68/05, A/BIM/46/95, A/GUM/33/84, A/ORS/66/84, A/ORS/75/88, A/TNAn/60/947/Asia/1, A/IRN/05, Asia/IRN/05, O/HK/2001, 0/UKG/3952/2001, O/UKG/4141/2001, Asia 1/HNK/CHA/05, Asia I/XJ, HK/70, O/UKG/7039/2001, O/UKG/9161/2001, O/UKG/7299/2001, O/UKG/4014/2001, O/UKG/4998/2001, O/UKG/9443/2001, O/UKG/5470/2001, O/UKG/5681/2001, O/ES/2001, HKN/2002, 05India, O/BKF/2/92, K/37/84/A, KEN/1/76/A, GAM/51/98/A, A10/Holland, O/KEN/1/91, O/IND49/97, O/IND65/98, O/IND64/98, O/IND48/98, O/IND47/98, O/IND82/97, O/IND81/99, O/IND81/98, O/IND79/97, O/IND78/97, O/IND75/97, O/IND74/97, O/IND70/97, O/IND66/98, O/IND63/97, O/IND61/97, O/IND57/98, O/IND56/98, O/IND55/98, O/IND54/98, O/IND469/98, O/IND465/97, O/IND464/97, O/IND424/97, O/IND423/97, O/IND420/97, O/IND414/97, O/IND411/97, O/IND410/97, O/IND409/97, O/IND407/97, O/IND399/97, O/IND39/97, O/IND391/97, O/IND38/97, O/IND384/97, O/IND380/97, O/IND37/97, O/IND352/97, O/IND33/97, O/IND31/97, O/IND296/97, O/IND23/99, O/IND463/97, O/IND461/97, O/IND427/98, O/IND28/97, O/IND287/99, O/IND285/99, O/IND282/99, O/IND281/97, O/IND27/97, O/IND278/97, O/IND256/99, O/IND249/99, O/IND210/99, O/IND208/99, O/IND207/99, O/IND205/99, O/IND185/99, O/IND175/99, O/IND170/97, O/IND164/99, O/IND160/99, O/IND153/99, O/IND148/99, O/IND146/99, O/IND/R2/75, O/SKR/2000, A22/India/17/77 and any combination thereof.
Item 25. The vaccine according to any one of items 1-24 wherein the antigen is an inactivated FMD virus.
Item 26. The vaccine according to item 24, wherein the inactivated FMD virus is a recombinant virus.
Item 27. The vaccine according to item 26 wherein the recombinant FMD virus lacks functional leader protein.
Item 28. The vaccine according to item 26 or 27 wherein the recombinant FMD virus further comprises one or more DIVA markers.
Item 29. The vaccine according to item 26 wherein the recombinant FMD virus further comprises one or more deletion sequences as negative markers.
Item 30. The vaccine according to item 26 wherein the recombinant FMD virus further comprises one or mutations in capsid coding sequences inducing stability.
Item 31. The vaccine according to any one of items 26-30, wherein the recombinant FMD virus is expresses a capsid from a heterologous FMD strain.
Item 32. The vaccine according to item 31, wherein the heterologous FMD strain is a strain of serotype A, or serotype O, or serotype Asia, or serotype C, or serotype SAT1, or serotype SAT2 and/or serotype SAT 3.
Item 33. The vaccine according to item 31, wherein the heterologous FMD strain is a strain of serotype A, or serotype O, and/or serotype Asia 1.
Item 34. The vaccine according to item 31, wherein the heterologous FMD strain is a strain of serotype A, or serotype O, and/or serotype C.
Item 35. The vaccine according to item 31, wherein the heterologous FMD strain is a strain of serotype SAT1, or serotype SAT2 and/or serotype SAT 3.
Item 36. The vaccine according to item 31, wherein the heterologous strain is selected from the group consisting of O1 Manisa, O1 BFS or Campos, Asia 1 Shamir, A Iran '96, A22 Iraq, SAT2 Saudi Arabia, A24/Cruzeiro, A10-61, A5, A12, C3/Indaial, O1, C1-Santa Pau, C1-C5, A22/550/Azerbaijan/65, SAT1-SAT3, A, A/TNC/71/94, A/IND/2/68, A/IND/3/77, A/IND/5/68, A/IND/7/82, A/IND/16/82, A/IND/17/77, A/IND/17/82, A/IND/19/76, A/IND/20/82, A/IND/22/82, A/IND/25/81, A/IND/26/82, A/IND/54/79, A/IND/57/79, A/IND/73/79, A/IND/85/79, A/IND/86/79, A/APA/25/84, A/APN/41/84, A/APS/44/05, A/APS/50/05, A/APS/55/05, A/APS/66/05, A/APS/68/05, A/BIM/46/95, A/GUM/33/84, A/ORS/66/84, A/ORS/75/88, A/TNAn/60/947/Asia/1, A/IRN/05, Asia/IRN/05, O/HK/2001, 0/UKG/3952/2001, O/UKG/4141/2001, Asia 1/HNK/CHA/05, Asia I/XJ, HK/70, O/UKG/7039/2001, O/UKG/9161/2001, O/UKG/7299/2001, O/UKG/4014/2001, O/UKG/4998/2001, O/UKG/9443/2001, O/UKG/5470/2001, O/UKG/5681/2001, O/ES/2001, HKN/2002, 05India, O/BKF/2/92, K/37/84/A, KEN/1/76/A, GAM/51/98/A, A10/Holland, O/KEN/1/91, O/IND49/97, O/IND65/98, O/IND64/98, O/IND48/98, O/IND47/98, O/IND82/97, O/IND81/99, O/IND81/98, O/IND79/97, O/IND78/97, O/IND75/97, O/IND74/97, O/IND70/97, O/IND66/98, O/IND63/97, O/IND61/97, O/IND57/98, O/IND56/98, O/IND55/98, O/IND54/98, O/IND469/98, O/IND465/97, O/IND464/97, O/IND424/97, O/IND423/97, O/IND420/97, O/IND414/97, O/IND411/97, O/IND410/97, O/IND409/97, O/IND407/97, O/IND399/97, O/IND39/97, O/IND391/97, O/IND38/97, O/IND384/97, O/IND380/97, O/IND37/97, O/IND352/97, O/IND33/97, O/IND31/97, O/IND296/97, O/IND23/99, O/IND463/97, O/IND461/97, O/IND427/98, O/IND28/97, O/IND287/99, O/IND285/99, O/IND282/99, O/IND281/97, O/IND27/97, O/IND278/97, O/IND256/99, O/IND249/99, O/IND210/99, O/IND208/99, O/IND207/99, O/IND205/99, O/IND185/99, O/IND175/99, O/IND170/97, O/IND164/99, O/IND160/99, O/IND153/99, O/IND148/99, O/IND146/99, O/SKR/2000, A22/India/17/77, O/IND/R2/75 and any combination thereof.
Item 37. The vaccine according to any one of items 24-36 wherein the FMD virus is present in the amount of at least 4 μg per dose per strain.
Item 38. The vaccine according to any one of items 24-37 wherein the FMD virus is present in the amount of at least 8 μg per dose per strain.
Item 39. The vaccine according to any one of items 24-38 wherein the FMD virus is present in the amount of about 10 μg per dose per strain.
Item 40. The vaccine according to any one of items 1-39 wherein the ruminant is a bovine.
Item 41. Use of a vaccine comprising an FMD antigen and an adjuvant comprising (or consisting of or consisting essentially of) oil, a polycationic carrier, and an immunostimulatory oligonucleotide, wherein said vaccine is a water-in-oil (W/O) emulsion, for use in preventing foot lesions caused by FMD infection in a ruminant, wherein said vaccine is administered as a single prime dose and one or more booster doses, wherein the first of said one or more booster doses is administered 7 to about 12 months after the single prime dose.
Item 42. The use according to item 41, wherein each consequent booster dose is administered 7 to about 12 months after the previous booster dose.
Item 43. The use according to item 41 or 42, wherein said first booster dose is administered about 8, or about 9, or about 10, or about 11 or about 12 months after the single prime dose.
Item 44. The use according to any one of item 41-43 wherein the cationic carrier is DEAE Dextran.
Item 45. The use according to item 44, wherein the DEAE Dextran is present in the amount of 25-250 mg per dose.
Item 46. The use according to item 45, wherein the immunostimulatory oligonucleotide is present in the amount of at least 25 μg per dose.
Item 47. The use according to item 46, wherein the immunostimulatory oligonucleotide is present in the amount of 50-150 μg per dose.
Item 48. The use according to any one of items 41-46, wherein the immunostimulatory oligonucleotide is a P-class immunostimulatory oligonucleotide.
Item 49. The use according to item 48, wherein the P-class immunostimulatory oligonucleotide is a modified P-class immunostimulatory oligonucleotide.
Item 50. The use according to item 49, wherein the modified P-class immunostimulatory oligonucleotide comprises 5′-lodo-2′-deoxyuridine or 5-Ethyl-2′-deoxyuridine.
Item 51. The use according to item 50, wherein the P-class immunostimulatory oligonucleotide comprises 15 consecutive oligonucleotides of SEQ ID NO: 8.
Item 52. The use according to item 51, wherein the P-class immunostimulatory oligonucleotide comprises 20 consecutive oligonucleotides of SEQ ID NO: 8.
Item 53. The use according to item 52, wherein the P-class immunostimulatory oligonucleotide comprises SEQ ID NO: 8.
Item 54. The use according to any one of items 41-53 wherein the vaccine comprises at least 40% oil v/v.
Item 55. The use according to item 54 wherein the vaccine comprises at least 45% oil v/v.
Item 56. The use according to item 55 wherein the vaccine comprises at least 48% oil v/v.
Item 57. The use according to item 56, wherein the vaccine comprises at least 52% oil v/v.
Item 58. The use according to item 57, wherein the vaccine comprises at least 60% oil v/v.
Item 59. The use according to any one of items 41-58 wherein the oil is a non-metabilolizable oil.
Item 60. The use according to item 59, wherein the non-metabolizable oil is a light mineral oil.
Item 61. The use according to any one of items 41-60 wherein the FMD antigen is from an FMD virus of Type O, A and/or Asia Type 1.
Item 62. The use according to any one of items 41-60 wherein the FMD antigen is from an FMD virus of Type O, A and/or C serotype.
Item 63. The use according to any one of items 41-60 wherein the FMD antigen is from an FMD virus of SAT-1, SAT/2 and/or SAT3 serotype.
Item 64. The use according to item 41-63, wherein the FMD antigen is from a strain selected from the group consisting of O1 Manisa, O1 BFS or Campos, Asia 1 Shamir, A Iran '96, A22 Iraq, SAT2 Saudi Arabia, A10-61, A5, A12, A24/Cruzeiro, C3/Indaial, O1, C1-Santa Pau, C1-C5, A22/550/Azerbaijan/65, SAT1-SAT3, A, A/TNC/71/94, A/IND/2/68, A/IND/3/77, A/IND/5/68, A/IND/7/82, A/IND/16/82, A/IND/17/77, A/IND/17/82, A/IND/19/76, A/IND/20/82, A/IND/22/82, A/IND/25/81, A/IND/26/82, A/IND/54/79, A/IND/57/79, A/IND/73/79, A/IND/85/79, A/IND/86/79, A/APA/25/84, A/APN/41/84, A/APS/44/05, A/APS/50/05, A/APS/55/05, A/APS/66/05, A/APS/68/05, A/BIM/46/95, A/GUM/33/84, A/ORS/66/84, A/ORS/75/88, A/TNAn/60/947/Asia/1, A/IRN/05, Asia/IRN/05, O/HK/2001, 0/UKG/3952/2001, O/UKG/4141/2001, Asia 1/HNK/CHA/05, Asia I/XJ, HK/70, O/UKG/7039/2001, O/UKG/9161/2001, O/UKG/7299/2001, O/UKG/4014/2001, O/UKG/4998/2001, O/UKG/9443/2001, O/UKG/5470/2001, O/UKG/5681/2001, O/ES/2001, HKN/2002, O5India, O/BKF/2/92, K/37/84/A, KEN/1/76/A, GAM/51/98/A, A10/Holland, O/KEN/1/91, O/IND49/97, O/IND65/98, O/IND64/98, O/IND48/98, O/IND47/98, O/IND82/97, O/IND81/99, O/IND81/98, O/IND79/97, O/IND78/97, O/IND75/97, O/IND74/97, O/IND70/97, O/IND66/98, O/IND63/97, O/IND61/97, O/IND57/98, O/IND56/98, O/IND55/98, O/IND54/98, O/IND469/98, O/IND465/97, O/IND464/97, O/IND424/97, O/IND423/97, O/IND420/97, O/IND414/97, O/IND411/97, O/IND410/97, O/IND409/97, O/IND407/97, O/IND399/97, O/IND39/97, O/IND391/97, O/IND38/97, O/IND384/97, O/IND380/97, O/IND37/97, O/IND352/97, O/IND33/97, O/IND31/97, O/IND296/97, O/IND23/99, O/IND463/97, O/IND461/97, O/IND427/98, O/IND28/97, O/IND287/99, O/IND285/99, O/IND282/99, O/IND281/97, O/IND27/97, O/IND278/97, O/IND256/99, O/IND249/99, O/IND210/99, O/IND208/99, O/IND207/99, O/IND205/99, O/IND185/99, O/IND175/99, O/IND170/97, O/IND164/99, O/IND160/99, O/IND153/99, O/IND148/99, O/IND146/99, O/SKR/2000, A22/India/17/77 and any combination thereof.
Item 65. The use according to any one of items 41-64 wherein the antigen is an inactivated FMD virus.
Item 66. The use according to item 64, wherein the inactivated FMD virus is a recombinant virus.
Item 67. The use according to item 66 wherein the recombinant FMD virus lacks functional leader protein.
Item 68. The use according to item 66 or 67 wherein the recombinant FMD virus further comprises one or more DIVA markers.
Item 69. The use according to any one of items 66-68, wherein the recombinant FMD virus is expresses a capsid from a heterologous FMD strain.
Item 70. The use according to item 69, wherein the heterologous FMD strain is a strain of serotype A, or serotype O, or serotype Asia, or serotype C, or serotype SAT1, or serotype SAT2 and/or serotype SAT 3.
Item 71. The use according to item 69, wherein the heterologous FMD strain is a strain of serotype A, or serotype O, and/or serotype Asia 1.
Item 72. The use according to item 69, wherein the heterologous FMD strain is a strain of serotype A, or serotype O, and/or serotype C.
Item 73. The use according to item 69, wherein the heterologous FMD strain is a strain of serotype SAT1, or serotype SAT2 and/or serotype SAT 3.
Item 74. The use according to item 69, wherein the heterologous strain is selected from the group consisting of O1 Manisa, O1 BFS or Campos, Asia 1 Shamir, A Iran '96, A22 Iraq, SAT2 Saudi Arabia, A24/Cruzeiro, A10-61, A5, A12, C3/Indaial, O1, C1-Santa Pau, C1-C5, A22/550/Azerbaijan/65, SAT1-SAT3, A, A/TNC/71/94, A/IND/2/68, A/IND/3/77, A/IND/5/68, A/IND/7/82, A/IND/16/82, A/IND/17/77, A/IND/17/82, A/IND/19/76, A/IND/20/82, A/IND/22/82, A/IND/25/81, A/IND/26/82, A/IND/54/79, A/IND/57/79, A/IND/73/79, A/IND/85/79, A/IND/86/79, A/APA/25/84, A/APN/41/84, A/APS/44/05, A/APS/50/05, A/APS/55/05, A/APS/66/05, A/APS/68/05, A/BIM/46/95, A/GUM/33/84, A/ORS/66/84, A/ORS/75/88, A/TNAn/60/947/Asia/1, A/IRN/05, Asia/IRN/05, O/HK/2001, 0/UKG/3952/2001, O/UKG/4141/2001, Asia 1/HNK/CHA/05, Asia I/XJ, HK/70, O/UKG/7039/2001, O/UKG/9161/2001, O/UKG/7299/2001, O/UKG/4014/2001, O/UKG/4998/2001, O/UKG/9443/2001, O/UKG/5470/2001, O/UKG/5681/2001, O/ES/2001, HKN/2002, 05India, O/BKF/2/92, K/37/84/A, KEN/1/76/A, GAM/51/98/A, A10/Holland, O/KEN/1/91, O/IND49/97, O/IND65/98, O/IND64/98, O/IND48/98, O/IND47/98, O/IND82/97, O/IND81/99, O/IND81/98, O/IND79/97, O/IND78/97, O/IND75/97, O/IND74/97, O/IND70/97, O/IND66/98, O/IND63/97, O/IND61/97, O/IND57/98, O/IND56/98, O/IND55/98, O/IND54/98, O/IND469/98, O/IND465/97, O/IND464/97, O/IND424/97, O/IND423/97, O/IND420/97, O/IND414/97, O/IND411/97, O/IND410/97, O/IND409/97, O/IND407/97, O/IND399/97, O/IND39/97, O/IND391/97, O/IND38/97, O/IND384/97, O/IND380/97, O/IND37/97, O/IND352/97, O/IND33/97, O/IND31/97, O/IND296/97, O/IND23/99, O/IND463/97, O/IND461/97, O/IND427/98, O/IND28/97, O/IND287/99, O/IND285/99, O/IND282/99, O/IND281/97, O/IND27/97, O/IND278/97, O/IND256/99, O/IND249/99, O/IND210/99, O/IND208/99, O/IND207/99, O/IND205/99, O/IND185/99, O/IND175/99, O/IND170/97, O/IND164/99, O/IND160/99, O/IND153/99, O/IND148/99, O/IND146/99, O/SKR/2000, A22/India/17/77 and any combination thereof.
Item 75. The use according to any one of items 64-74 wherein the FMD virus is present in the amount of at least 4 μg per dose per strain.
Item 76. The use according to any one of items 64-75 wherein the FMD virus is present in the amount of at least 8 μg per dose per strain.
Item 77. The use according to any one of items 64-76 wherein the FMD virus is present in the amount of about 10 μg per dose per strain.
Item 78. The use according to any one of items 41-77 wherein the ruminant is a bovine.
Item 79. The use according to any one of items 41-78, wherein the ruminant is FMD-negative.
Item 80. The vaccine according to any one of claims 1-40, wherein the ruminant is FMD-negative.

The following examples are presented as illustrative embodiments but should not be taken as limiting the scope of the invention. Many changes, variations, modifications, and other uses and applications of this invention will be apparent to those skilled in the art.

### EXAMPLES

### Example 1: Single-Dose Vaccine Provides One-Year Duration of Immunity

The objective of the study was to evaluate 1 year duration of Immunity (DOI) of a trivalent inactivated FMDV vaccine adjuvanted with an optimized Zoetis adjuvant formulation for improved protective immunity in Indian cattle against challenge with homologous type O FMD virus.

Seronegative to FMD (SN titer <1:8) six to twelve months old male cattle (Bos indicus) weighting 100-200 kg were used for this study. All animals were healthy at the time of vaccination. During the vaccination stage, the animals were kept in a non-containment animal experimentation facility, as mandated by the local government authorities. Animals were housed in one shed. During the challenge phase, vaccinated and unvaccinated calves were housed in secure, containment animal experimentation facility as mandated by the local government authorities. Animals were housed in five identical rooms. In each room 8 animals were housed with 2 animals per treatment group per room. Animals were transported to the challenge facility on day 358 prior to challenge. Water and other feed ingredients were fed as per requirements established by the local authorities.

The animals were acclimated at least two weeks from the date of arrival at the test facility. The animals were dewormed with Panacur Vet (Fenbendazole 2.5% suspension, M/s Intervet) 5 mg/Kg body weight for each animal.

The treatments are provided in table 2.

The animals were vaccinated on day zero by intramuscular injection of 2 ml of the vaccine appropriate for the group.

Blood Collection for serology: Blood samples were collected for detection of antibody responses to the three antigens using a sterile syringe and needle on day-14, 0, 29, 60, 90, 120, 149, 180, 210, 240, 270, 300, 330, 365 and 379 of study from all calves in all treatment groups. Calves from all groups were bled via the jugular vein, and 8-10 ml blood were collected using vacutainers.

TABLE 2

| Group | N | Vaccine | Composition |
|---|---|---|---|
| T01 | 10 | None | Saline |
| T02 | 10 | Commercial | Proprietary to manufacturer. |
| | | | Contains FMD-A, |
| | | | FMD-O, FMD-Asia 1 |
| T03 | 10 | Experimental vaccine | FMD-A (5 μg/dose), |
| | | Normal AG dose | FMD-O (8 μg/dose), |
| | | | FMD-Asia 1 (5 μg/dose) + Adjuvant |
| T04 | 10 | Experimental vaccine | FMD-A (10 μg/dose), |
| | | High AG dose | FMD-O (16 μg/dose), |
| | | | FMD-Asia 1 (8 μg/dose) + Adjuvant |

The adjuvant used in T03 and T04 was identical and contained (per 2 ml dose): Light mineral oil 45% v/v, SPAN®80 6.3% v/v; DEAE Dextran 100 mg; CpG 50 μg; TWEEN® 80 1.45% v/v.

Heparinized whole blood samples to test for cell-mediated immune responses (IFN gamma ELISPOT Assay) were also collected using sterile syringe and needle on day 0 (pre-vaccination), 9, 29, 180 and 330 from all calves in all treatment groups.

Challenge: On Study day 365 animals were challenged. Animals were transported to the challenge facility one week prior to challenge day. Challenge dose: 10000 BID50 (median bovine infectivity dose in 0.1 mL volume) of cattle adapted homologous FMD type O virus by intra-dermolingual inoculation.

Post challenge observations were observed and documented during the 10-day post-challenge period.

Serum neutralization test (SNT) is based on neutralization of virus infection of target cells BHK21 using antibody from animals. The SNT was performed similarly to the test described by Crowther et al. (1984). Briefly, dilutions of sera were mixed with 100 TCID50 of either A/IND/40/00, O/IND/R2/75 or Asia-1/IND/63/72 in microtitre plates and incubated for 1 hour at 37° C. Thereafter, an equal volume of BHK21 cells at 3×10⁶ cells/ml was added and the plates further incubated at 37° C. The reduction in virus-specific cytopathic effect (cpe) was read after 48 hours, and the virus neutralization titre of the serum calculated by the Reed and Muench method.

Final study outcome was based on the protection against development of foot lesions, against FMD Virus serotype “O”.

Post challenge development of foot lesions was monitored and recorded. Presence or absence of foot lesions was considered significant (primary variable) and used to define the efficacy of the various vaccines. Because foot lesions were at a distance from where the challenge was administered their presence meant that the virus was able to spread from the tongue site to the feet. Tongue lesions (secondary variable) were scored on day 367 and foot lesions on day 375. Thus, the primary variable (foot lesions) are secondary lesions and the secondary variable (tongue lesions) is the primary lesions.

Whereas 100% of the control T01 animals developed tongue (primary lesions) and foot lesions (secondary lesions), 100% of the T02 (positive control) animals developed tongue lesions and 60% developed foot lesions. In contrast, 60% of the T03 (normal dose vaccine) and T04 (high dose vaccine) animals developed tongue lesions, but no foot lesions (0%) were observed in T03 group animals and only 10% of T04 (1/10) developed foot lesions (Table 3).

TABLE 3
Summary of Lesions

| | | TREATMENT GROUP | | | | | | | |
|---|---|---|---|---|---|---|---|---|---|
| | | | | T02: | | | | | |
| | | | | Positive | | | | | |
| | | | | Control | | T03: FMD | | T04: FMD | |
| | | T01: | | Single Dose | | Trivalent | | Trivalent | |
| | | Negative | | (Commercial | | vaccine- | | vaccine- | |
| | | Control | | FMD | | Normal | | High | |
| | | (Saline) | | vaccine) | | Dose | | Dose | |
| | | N | % | N | % | n | % | n | % |
| LESION TYPE | LESION | | | | | | | | |
| | PRESENT | | | | | | | | |
| PRIMARY (TONGUE LESIONS) | No | 0 | 0.0 | 0 | 0.0 | 4 | 40.0 | 4 | 40.0 |
| | Yes | 10 | 100.0 | 10 | 100.0 | 6 | 60.0 | 6 | 60.0 |
| SECONDARY (FOOT LESIONS) | No | 0 | 0.0 | 4 | 40.0 | 10 | 100.0 | 9 | 90.0 |
| | Yes | 10 | 100.0 | 6 | 60.0 | 0 | 0.0 | 1 | 10.0 |
| EITHER (TONGUE OR FOOT | No | 0 | 0.0 | 0 | 0.0 | 4 | 40.0 | 4 | 40.0 |
| LESIONS) | Yes | 10 | 100.0 | 10 | 100.0 | 6 | 60.0 | 6 | 60.0 |

Significantly fewer animals in the T03 and T04 groups developed foot lesions compared with the T01 control group (p<0.0001; Table 4). In contrast, the T02 group was not significantly different from the T01 control group (p=0.0867; Table 4). Furthermore, the proportion of animals that developed foot lesions in the T03 group was significantly lower than the T02 group (p=0.0108) and there was significant no difference between the T04 and T02 groups for foot lesions (p=0.0573; Table 4), although numerically the T04 group had 1 of 10 animals and T02 had 6 of 10 animals positive.

TABLE 4
Analysis of Foot Lesions:
Primary Outcome Variable

| treatment | |
|---|---|
| comparison | P-value |
| Overall | <0.0001 |
| T01 vs. T02 | 0.0867 |
| T01 vs. T03 | <0.0001 |
| T01 vs. T04 | 0.0001 |
| T02 vs. T03 | 0.0108 |
| T02 vs. T04 | 0.0573 |
| T03 vs. T04 | 1.0000 |

There were no significant pairwise differences between any of the groups in terms of the proportion of animals with tongue lesions (p>0.05) or the proportion of animals with any lesion (foot or tongue, p>0.05). Nevertheless, groups T03 and T04 were numerically different from groups T01 and/or T02 (p=0.0867).

The geometric means of the SN titers against the O antigen for T02 through T04 are summarized in Tables 5-7.

The SN was below 8 in all animals in group T01 pre-challenge and had risen to 256 on day 379 (95% confidence interval 183.9 to 356.3). Animals with titer 8 and above were considered seropositive. Animals with titer below 8 were considered seronegative.

All but one T02 animal responded to vaccination. Five of 9 responding calves became sero-negative between day 180 to 210, 4 had persistent titers till day 365 (3 of these 4 were negative for foot lesions). In contrast, all T03 and T04 animals sero-converted and developed titers which persisted till day 365.

The analysis of LSM of SN Titers to O antigen (log transformed) are summarized in Table 8. The summary shows that T03 and T04 group animals developed significantly higher geometric mean titers (p<0.05) on multiple days of analysis compared to the positive control group T02. There were no significant differences between the geometric mean titers of T03 and T04 at any time prior to challenge. The SN titers of the T02 through T04 groups on day 379, 14 days after challenge were significantly higher than the SN titers of the T01 control animals (p<0.05). There were no differences between the day 379 SN titers of T02 through T04.

TABLE 5
Geometric Means of SN Titers of T02 Positive Control Group Animals to O Antigen

| | | | lower 95% | upper 95% | | |
|---|---|---|---|---|---|---|
| day of | no. | geometric | conf. limit | conf. limit | | |
| study | animals | mean | for mean | for mean | minimum | Maximum |
| 14 | 10 | 70.8 | 41.2 | 121.9 | 4 | 256 |
| 29 | 10 | 36.9 | 23.3 | 58.3 | 4 | 128 |
| 60 | 10 | 21.9 | 13.3 | 36.1 | 4 | 90 |
| 90 | 10 | 19.0 | 11.3 | 32.0 | 4 | 45 |
| 120 | 10 | 21.1 | 12.3 | 36.2 | 8 | 45 |
| 149 | 10 | 16.5 | 9.7 | 28.0 | 4 | 45 |
| 180 | 10 | 6.7 | 4.1 | 11.1 | 4 | 23 |
| 210 | 10 | 6.7 | 4.4 | 10.1 | 4 | 23 |
| 240 | 10 | 6.1 | 3.9 | 9.5 | 4 | 16 |
| 270 | 10 | 7.7 | 4.5 | 13.3 | 4 | 23 |
| 300 | 10 | 8.0 | 5.0 | 12.9 | 4 | 23 |
| 330 | 10 | 7.0 | 4.1 | 11.9 | 4 | 16 |
| 365 | 10 | 7.2 | 4.4 | 11.8 | 4 | 23 |
| 379 | 10 | 954.4 | 860.5 | 1060.7 | 724 | 1024 |

TABLE 6
Geometric Means of SN Titers of T03 Experimental Vaccine
Normal AG Dose Group Animals to O Antigen

| | | | lower | upper | | |
|---|---|---|---|---|---|---|
| | | | 95% | 95% | | |
| | | | conf. | conf. | | |
| day of | no. | geometric | limit | limit | | |
| study | animals | mean | for mean | for mean | minimum | maximum |
| 14 | 10 | 141.8 | 82.4 | 244.0 | 64 | 256 |
| 29 | 10 | 87.2 | 55.2 | 137.7 | 45 | 128 |
| 60 | 10 | 78.7 | 47.7 | 129.7 | 32 | 181 |
| 90 | 10 | 78.8 | 46.8 | 132.7 | 23 | 181 |
| 120 | 10 | 73.6 | 43.0 | 125.9 | 16 | 181 |
| 149 | 10 | 61.6 | 36.3 | 104.6 | 16 | 181 |
| 180 | 10 | 73.3 | 44.4 | 121.0 | 32 | 181 |
| 210 | 10 | 59.6 | 39.3 | 90.2 | 23 | 128 |
| 240 | 10 | 61.6 | 39.3 | 96.7 | 32 | 181 |
| 270 | 10 | 73.3 | 42.6 | 126.0 | 32 | 256 |
| 300 | 10 | 66.0 | 40.9 | 106.3 | 45 | 181 |
| 330 | 10 | 73.3 | 43.1 | 125.0 | 32 | 181 |
| 365 | 10 | 70.9 | 43.2 | 116.2 | 32 | 181 |
| 379 | 10 | 831.7 | 607.8 | 1138.1 | 256 | 1024 |

TABLE 7
Geometric Means of SN Titers of T04 Experimental Vaccine High
AG Dose Group Animals to O Antigen

| | | | lower | upper | | |
|---|---|---|---|---|---|---|
| | | | 95% | 95% | | |
| | | | conf. | conf. | | |
| day of | no. | geometric | limit | limit | | |
| study | animals | mean | for mean | for mean | minimum | maximum |
| 14 | 10 | 187.3 | 108.8 | 322.3 | 64 | 362 |
| 29 | 10 | 107.5 | 68.0 | 169.9 | 64 | 181 |
| 60 | 10 | 127.7 | 77.5 | 210.5 | 45 | 512 |
| 90 | 10 | 103.8 | 61.7 | 174.8 | 23 | 362 |
| 120 | 10 | 78.7 | 46.0 | 134.7 | 16 | 362 |
| 149 | 10 | 73.7 | 43.4 | 125.1 | 23 | 362 |
| 180 | 10 | 59.7 | 36.2 | 98.6 | 8 | 256 |
| 210 | 10 | 55.5 | 36.7 | 84.1 | 8 | 128 |
| 240 | 10 | 57.7 | 36.8 | 90.5 | 8 | 181 |
| 270 | 10 | 70.7 | 41.1 | 121.6 | 11 | 256 |
| 300 | 10 | 55.6 | 34.5 | 89.6 | 11 | 256 |
| 330 | 10 | 61.8 | 36.3 | 105.4 | 8 | 256 |
| 365 | 10 | 59.5 | 36.3 | 97.6 | 11 | 256 |
| 379 | 10 | 955.4 | 860.5 | 1060.7 | 724 | 1024 |

TABLE 8
LEAST SQUARES MEAN DIFFERENCES BETWEEN TREATMENT GROUPS AND
95% CONFIDENCE INTERVALS TO O ANTIGEN TITERS (Log Transformed)

| | | least | | lower 95% | upper 95% | |
|---|---|---|---|---|---|---|
| | | squares | | conf. limit | conf. limit | |
| treatment | day of | mean | standard | for mean | for mean | |
| comparison | study | diff. | error | difference | difference | P-value |
| T02-T03 | 14 | −0.694 | 0.374 | −1.462 | 0.074 | 0.0747 |
| T02-T04 | 14 | −0.972 | 0.374 | −1.740 | −0.204 | 0.0150 |
| T03-T04 | 14 | −0.278 | 0.374 | −1.046 | 0.489 | 0.4634 |
| T02-T03 | 29 | −0.860 | 0.315 | −1.507 | −0.214 | 0.0110 |
| T02-T04 | 29 | −1.070 | 0.315 | −1.717 | −0.423 | 0.0021 |
| T03-T04 | 29 | −0.210 | 0.315 | −0.856 | 0.437 | 0.5117 |
| T02-T03 | 60 | −1.278 | 0.345 | −1.985 | −0.571 | 0.0010 |
| T02-T04 | 60 | −1.763 | 0.345 | −2.469 | −1.056 | <0.0001 |
| T03-T04 | 60 | −0.485 | 0.345 | −1.192 | 0.222 | 0.1709 |
| T02-T03 | 90 | −1.422 | 0.359 | −2.158 | −0.685 | 0.0005 |
| T02-T04 | 90 | −1.698 | 0.359 | −2.435 | −0.961 | <0.0001 |
| T03-T04 | 90 | −0.276 | 0.359 | −1.013 | 0.461 | 0.4486 |
| T02-T03 | 120 | −1.247 | 0.371 | −2.008 | −0.487 | 0.0023 |
| T02-T04 | 120 | −1.315 | 0.371 | −2.075 | −0.554 | 0.0014 |
| T03-T04 | 120 | −0.068 | 0.371 | −0.828 | 0.693 | 0.8565 |
| T02-T03 | 149 | −1.317 | 0.365 | −2.065 | −0.568 | 0.0012 |
| T02-T04 | 149 | −1.496 | 0.365 | −2.244 | −0.747 | 0.0003 |
| T03-T04 | 149 | −0.179 | 0.365 | −0.928 | 0.569 | 0.6270 |
| T02-T03 | 180 | −2.387 | 0.346 | −3.096 | −1.678 | <0.0001 |
| T02-T04 | 180 | −2.182 | 0.346 | −2.891 | −1.473 | <0.0001 |
| T03-T04 | 180 | 0.205 | 0.346 | −0.504 | 0.914 | 0.5586 |
| T02-T03 | 210 | −2.185 | 0.286 | −2.772 | −1.598 | <0.0001 |
| T02-T04 | 210 | −2.115 | 0.286 | −2.702 | −1.528 | <0.0001 |
| T03-T04 | 210 | 0.070 | 0.286 | −0.517 | 0.657 | 0.8075 |
| T02-T03 | 240 | −2.319 | 0.310 | −2.955 | −1.682 | <0.0001 |
| T02-T04 | 240 | −2.253 | 0.310 | −2.889 | −1.616 | <0.0001 |
| T03-T04 | 240 | 0.066 | 0.310 | −0.571 | 0.703 | 0.8332 |
| T02-T03 | 270 | −2.249 | 0.374 | −3.016 | −1.483 | <0.0001 |
| T02-T04 | 270 | −2.214 | 0.374 | −2.980 | −1.447 | <0.0001 |
| T03-T04 | 270 | 0.036 | 0.374 | −0.731 | 0.802 | 0.9244 |
| T02-T03 | 300 | −2.111 | 0.329 | −2.786 | −1.436 | <0.0001 |
| T02-T04 | 300 | −1.940 | 0.329 | −2.615 | −1.265 | <0.0001 |
| T03-T04 | 300 | 0.171 | 0.329 | −0.504 | 0.846 | 0.6072 |
| T02-T03 | 330 | −2.354 | 0.367 | −3.108 | −1.601 | <0.0001 |
| T02-T04 | 330 | −2.184 | 0.367 | −2.937 | −1.430 | <0.0001 |
| T03-T04 | 330 | 0.171 | 0.367 | −0.583 | 0.924 | 0.6461 |
| T02-T03 | 365 | −2.283 | 0.341 | −2.983 | −1.584 | <0.0001 |
| T02-T04 | 365 | −2.109 | 0.341 | −2.809 | −1.409 | <0.0001 |
| T03-T04 | 365 | 0.174 | 0.341 | −0.525 | 0.874 | 0.6130 |
| T01-T02 | 379 | −1.317 | −0.153 | −1.655 | 0.979 | <0.0001 |
| T01-T03 | 379 | −1.178 | −0.201 | −1.602 | 0.755 | <0.0001 |
| T01-T04 | 379 | −1.317 | −0.153 | −1.655 | 0.979 | <0.0001 |
| T02-T03 | 379 | 0.139 | 0.146 | −0.183 | 0.460 | 0.3632 |
| T02-T04 | 379 | 0.000 | 0.065 | −0.137 | 0.137 | 1.0000 |
| T03-T04 | 379 | −0.139 | 0.146 | −0.460 | 0.183 | 0.3632 |

CMI responses were measured pre and post-vaccination by IFN gamma ELISPOT assay. Analysis of these responses are summarized in Table 9. There were significant differences (p<0.05) between the CMI responses of T01 versus T03 on days 0, 9 and 180 (p<0.05), versus T04 on days 9, 180 and 330 and versus T02 on day 180. There were significant differences (p<0.05) between the CMI responses of T02 versus T03 on day 9 and between groups T03 and T04 on day 180.

TABLE 9
LEAST SQUARES MEAN DIFFERENCES BETWEEN TREATMENT GROUPS AND
95% CONFIDENCE INTERVALS CMI Responses (Log Transformed)

| | | least | | lower 95% | upper 95% | |
|---|---|---|---|---|---|---|
| | | squares | | conf. limit | conf. limit | |
| treatment | day of | mean | standard | for mean | for mean | |
| comparison | study | diff. | error | difference | difference | P-value |
| T01-T02 | 0 | −0.056 | 0.110 | −0.278 | 0.166 | 0.6119 |
| T01-T03 | 0 | −0.236 | 0.110 | −0.458 | −0.014 | 0.0381 |
| T01-T04 | 0 | −0.069 | 0.110 | −0.292 | 0.153 | 0.5313 |
| T02-T03 | 0 | −0.180 | 0.110 | −0.402 | 0.042 | 0.1094 |
| T02-T04 | 0 | −0.013 | 0.110 | −0.236 | 0.209 | 0.9048 |
| T03-T04 | 0 | 0.167 | 0.110 | −0.056 | 0.389 | 0.1369 |
| T01-T02 | 9 | −0.302 | 0.217 | −0.742 | 0.138 | 0.1723 |
| T01-T03 | 9 | −0.747 | 0.217 | −1.187 | −0.306 | 0.0015 |
| T01-T04 | 9 | −0.523 | 0.217 | −0.963 | −0.082 | 0.0213 |
| T02-T03 | 9 | −0.445 | 0.217 | −0.885 | −0.004 | 0.0479 |
| T02-T04 | 9 | −0.220 | 0.217 | −0.661 | 0.220 | 0.3164 |
| T03-T04 | 9 | 0.224 | 0.217 | −0.216 | 0.664 | 0.3087 |
| T01-T02 | 29 | −0.094 | 0.229 | −0.559 | 0.370 | 0.6828 |
| T01-T03 | 29 | −0.166 | 0.229 | −0.630 | 0.299 | 0.4746 |
| T01-T04 | 29 | −0.270 | 0.229 | −0.734 | 0.195 | 0.2468 |
| T02-T03 | 29 | −0.071 | 0.229 | −0.536 | 0.393 | 0.7580 |
| T02-T04 | 29 | −0.175 | 0.229 | −0.640 | 0.289 | 0.4491 |
| T03-T04 | 29 | −0.104 | 0.229 | −0.569 | 0.360 | 0.6520 |
| T01-T02 | 180 | −0.361 | 0.143 | −0.652 | −0.071 | 0.0162 |
| T01-T03 | 180 | −0.594 | 0.143 | −0.885 | −0.304 | 0.0002 |
| T01-T04 | 180 | −0.300 | 0.143 | −0.590 | −0.009 | 0.0437 |
| T02-T03 | 180 | −0.233 | 0.143 | −0.523 | 0.058 | 0.1131 |
| T02-T04 | 180 | 0.062 | 0.143 | −0.229 | 0.352 | 0.6682 |
| T03-T04 | 180 | 0.295 | 0.143 | 0.004 | 0.585 | 0.0471 |
| T01-T02 | 330 | −0.121 | 0.125 | −0.375 | 0.132 | 0.3388 |
| T01-T03 | 330 | −0.231 | 0.125 | −0.485 | 0.023 | 0.0734 |
| T01-T04 | 330 | −0.321 | 0.125 | −0.575 | −0.067 | 0.0147 |
| T02-T03 | 330 | −0.109 | 0.125 | −0.363 | 0.144 | 0.3881 |
| T02-T04 | 330 | −0.199 | 0.125 | −0.453 | 0.055 | 0.1204 |
| T03-T04 | 330 | −0.090 | 0.125 | −0.344 | 0.164 | 0.4782 |

The single dose trivalent FMD experimental vaccines administered to T03 group animals were significantly more efficacious than the positive control Commercial vaccine sold in India (p<0.05). The T04 vaccine did not induce a statistically significant protection compared to T02 vaccine but numerically only 1 of 10 T04 group calves were positive for foot lesions whereas 6 of 10 T02 group calves were positive (p=0.0573).

Based on the primary variable, foot lesions the T03 and T04 vaccines provided a 1-year DOI against challenge with the O strain.

Similarly, antibody responses to A type antigen in the T02 group were lower and 7 of 10 animals were sero-negative by day 365, whereas the titers were higher and persisted up to day 365 in the T03 and T04 groups. The results for groups T02-T04 are provided in tables 10-12. The data for T01 are not shown as all animals in T01 had SN below 8 until day 365.

TABLE 10
T02 Anti-A antigen SN Titers Geometric Means-Positive
Control Vaccine

| | | | lower | upper | | |
|---|---|---|---|---|---|---|
| | | | 95% | 95% | | |
| | | | conf. | conf. | | |
| day of | no. | geometric | limit | limit | | |
| study | animals | mean | for mean | for mean | minimum | maximum |
| 14 | 10 | 29.9 | 20.0 | 44.6 | 11 | 90 |
| 29 | 10 | 19.0 | 11.1 | 32.6 | 8 | 64 |
| 60 | 10 | 13.5 | 7.1 | 25.7 | 4 | 32 |
| 90 | 10 | 20.9 | 14.5 | 30.2 | 8 | 64 |
| 120 | 10 | 25.1 | 18.4 | 34.2 | 11 | 64 |
| 149 | 10 | 20.4 | 14.3 | 29.0 | 8 | 45 |
| 180 | 10 | 10.5 | 6.6 | 16.9 | 4 | 23 |
| 210 | 10 | 11.3 | 7.0 | 18.2 | 4 | 32 |
| 240 | 10 | 11.3 | 7.0 | 18.2 | 4 | 45 |
| 270 | 10 | 10.5 | 6.5 | 16.9 | 4 | 45 |
| 300 | 10 | 9.2 | 5.6 | 15.0 | 4 | 64 |
| 330 | 10 | 7.5 | 4.5 | 12.6 | 4 | 64 |
| 365 | 10 | 7.5 | 4.5 | 12.4 | 4 | 64 |

TABLE 11
T03 Anti-A antigen SN Titers Geometric Means-Normal
AG Dose Experimental Vaccine

| | | | lower | upper | | |
|---|---|---|---|---|---|---|
| | | | 95% | 95% | | |
| | | | conf. | conf. | | |
| day of | no. | geometric | limit | limit | | |
| study | animals | mean | for mean | for mean | minimum | maximum |
| 14 | 10 | 93.5 | 62.6 | 139.6 | 32 | 256 |
| 29 | 10 | 57.5 | 33.5 | 98.7 | 8 | 181 |
| 60 | 10 | 100.0 | 52.4 | 190.7 | 11 | 256 |
| 90 | 10 | 152.1 | 105.5 | 219.3 | 64 | 256 |
| 120 | 10 | 187.4 | 137.4 | 255.5 | 64 | 362 |
| 149 | 10 | 163.1 | 114.5 | 232.5 | 64 | 256 |
| 180 | 10 | 90.6 | 56.7 | 145.0 | 23 | 256 |
| 210 | 10 | 93.7 | 58.3 | 150.8 | 23 | 256 |
| 240 | 10 | 107.4 | 66.4 | 173.6 | 45 | 256 |
| 270 | 10 | 123.5 | 76.7 | 198.9 | 45 | 256 |
| 300 | 10 | 111.2 | 68.2 | 181.4 | 32 | 256 |
| 330 | 10 | 111.4 | 66.4 | 186.9 | 32 | 256 |
| 365 | 10 | 119.2 | 71.9 | 197.6 | 45 | 256 |

TABLE 12
T04 Anti-A antigen SN Titers Geometric Means-High
AG Dose Experimental Vaccine

| | | | lower | upper | | |
|---|---|---|---|---|---|---|
| | | | 95% | 95% | | |
| | | | conf. | conf. | | |
| day of | no. | geometric | limit | limit | | |
| study | animals | mean | for mean | for mean | minimum | maximum |
| 14 | 10 | 132.2 | 88.6 | 197.4 | 45 | 362 |
| 29 | 10 | 78.6 | 45.8 | 134.8 | 16 | 256 |
| 60 | 10 | 93.5 | 49.0 | 178.4 | 8 | 362 |
| 90 | 10 | 215.1 | 149.2 | 310.2 | 90 | 362 |
| 120 | 10 | 230.7 | 169.2 | 314.6 | 128 | 362 |
| 149 | 10 | 215.0 | 150.9 | 306.5 | 90 | 362 |
| 180 | 10 | 146.9 | 91.8 | 234.8 | 45 | 256 |
| 210 | 10 | 168.8 | 104.9 | 271.5 | 45 | 256 |
| 240 | 10 | 146.9 | 90.9 | 237.6 | 32 | 256 |
| 270 | 10 | 157.4 | 97.7 | 253.5 | 45 | 256 |
| 300 | 10 | 157.3 | 96.5 | 256.5 | 45 | 256 |
| 330 | 10 | 146.9 | 87.5 | 246.5 | 64 | 256 |
| 365 | 10 | 141.8 | 85.6 | 235.0 | 45 | 256 |

Statistical analysis of responses to antigen A are provided in Table 13.

TABLE 13
Statistical Analysis of A antigen Responses

| | | least | | lower 95% | upper 95% | |
|---|---|---|---|---|---|---|
| | | squares | | conf. limit | conf. limit | |
| treatment | day of | mean | standard | for mean | for mean | |
| comparison | study | diff. | error | difference | difference | P-value |
| T02-T03 | 14 | −1.142 | 0.276 | −1.708 | −0.575 | 0.0003 |
| T02-T04 | 14 | −1.488 | 0.276 | −2.055 | −0.922 | <0.0001 |
| T03-T04 | 14 | −0.347 | 0.276 | −0.913 | 0.220 | 0.2202 |
| T02-T03 | 29 | −1.109 | 0.372 | −1.872 | −0.345 | 0.0060 |
| T02-T04 | 29 | −1.420 | 0.372 | −2.183 | −0.657 | 0.0007 |
| T03-T04 | 29 | −0.311 | 0.372 | −1.075 | 0.452 | 0.4099 |
| T02-T03 | 60 | −2.004 | 0.445 | −2.917 | −1.091 | 0.0001 |
| T02-T04 | 60 | −1.937 | 0.445 | −2.851 | −1.024 | 0.0002 |
| T03-T04 | 60 | 0.066 | 0.445 | −0.847 | 0.980 | 0.8824 |
| T02-T03 | 90 | −1.983 | 0.252 | −2.500 | −1.465 | <0.0001 |
| T02-T04 | 90 | −2.329 | 0.252 | −2.847 | −1.812 | <0.0001 |
| T03-T04 | 90 | −0.347 | 0.252 | −0.864 | 0.171 | 0.1807 |
| T02-T03 | 120 | −2.011 | 0.214 | −2.449 | −1.572 | <0.0001 |
| T02-T04 | 120 | −2.219 | 0.214 | −2.657 | −1.780 | <0.0001 |
| T03-T04 | 120 | −0.208 | 0.214 | −0.646 | 0.231 | 0.3392 |
| T02-T03 | 149 | −2.080 | 0.244 | −2.581 | −1.579 | <0.0001 |
| T02-T04 | 149 | −2.356 | 0.244 | −2.857 | −1.855 | <0.0001 |
| T03-T04 | 149 | −0.276 | 0.244 | −0.777 | 0.225 | 0.2682 |
| T02-T03 | 180 | −2.151 | 0.324 | −2.815 | −1.488 | <0.0001 |
| T02-T04 | 180 | −2.634 | 0.324 | −3.298 | −1.970 | <0.0001 |
| T03-T04 | 180 | −0.483 | 0.324 | −1.146 | 0.181 | 0.1474 |
| T02-T03 | 210 | −2.117 | 0.328 | −2.789 | −1.445 | <0.0001 |
| T02-T04 | 210 | −2.705 | 0.328 | −3.377 | −2.033 | <0.0001 |
| T03-T04 | 210 | −0.588 | 0.328 | −1.260 | 0.084 | 0.0838 |
| T02-T03 | 240 | −2.255 | 0.331 | −2.935 | −1.575 | <0.0001 |
| T02-T04 | 240 | −2.569 | 0.331 | −3.248 | −1.889 | <0.0001 |
| T03-T04 | 240 | −0.314 | 0.331 | −0.993 | 0.366 | 0.3520 |
| T02-T03 | 270 | −2.464 | 0.328 | −3.138 | −1.790 | <0.0001 |
| T02-T04 | 270 | −2.707 | 0.328 | −3.380 | −2.033 | <0.0001 |
| T03-T04 | 270 | −0.243 | 0.328 | −0.916 | 0.431 | 0.4665 |
| T02-T03 | 300 | −2.495 | 0.337 | −3.186 | −1.803 | <0.0001 |
| T02-T04 | 300 | −2.841 | 0.337 | −3.533 | −2.150 | <0.0001 |
| T03-T04 | 300 | −0.347 | 0.337 | −1.038 | 0.345 | 0.3130 |
| T02-T03 | 330 | −2.699 | 0.357 | −3.432 | −1.967 | <0.0001 |
| T02-T04 | 330 | −2.976 | 0.357 | −3.708 | −2.244 | <0.0001 |
| T03-T04 | 330 | −0.277 | 0.357 | −1.009 | 0.456 | 0.4449 |
| T02-T03 | 365 | −2.768 | 0.348 | −3.482 | −2.053 | <0.0001 |
| T02-T04 | 365 | −2.941 | 0.348 | −3.655 | −2.227 | <0.0001 |
| T03-T04 | 365 | −0.173 | 0.348 | −0.888 | 0.541 | 0.6227 |

For SN titers to Asia 1 antigen, titers were still significantly higher in the T03 and T04 groups, but titers in T02 were more consistently induced and persisted up to day 365. Only one T02 animal was sero-negative on day 365. See tables 14-17. All animals in group T01 were seronegative throughout the study.

In all animals in groups T03 and T04 the SN titers to A and Asia 1 all animals were seropositive at 365 days post vaccination. The lowest SN titers to A type in groups T03 and T04 were 45 and 45, respectively, 365 days post-vaccination. The lowest titers to Asia 1 type in groups T03 and T04 were 45 and 64, respectively, 365 days post-vaccination.

These titers are sufficiently high to conclude that the animals would be protected against challenges with viruses of serotype A or Asia 1 365 days post-vaccination.

TABLE 14
T02 Anti-Asia Type 1 antigen SN Titers Geometric Means-Positive Control Vaccine

| | | | lower 95% | upper 95% | | |
|---|---|---|---|---|---|---|
| day of | no. | geometric | conf. limit | conf. limit | | |
| study | animals | mean | for mean | for mean | minimum | maximum |
| 14 | 10 | 137.0 | 88.6 | 211.9 | 64 | 362 |
| 29 | 10 | 50.1 | 27.3 | 92.1 | 8 | 362 |
| 60 | 10 | 24.3 | 14.0 | 42.1 | 4 | 128 |
| 90 | 10 | 36.8 | 21.0 | 64.3 | 4 | 181 |
| 120 | 10 | 26.0 | 13.7 | 49.1 | 4 | 128 |
| 149 | 10 | 27.8 | 14.7 | 52.7 | 4 | 128 |
| 180 | 10 | 45.2 | 25.7 | 79.4 | 11 | 256 |
| 210 | 10 | 41.9 | 23.9 | 73.3 | 8 | 181 |
| 240 | 10 | 43.5 | 27.1 | 69.6 | 11 | 181 |
| 270 | 10 | 23.4 | 13.5 | 40.5 | 4 | 90 |
| 300 | 10 | 35.5 | 21.0 | 59.9 | 8 | 256 |
| 330 | 10 | 29.7 | 17.0 | 52.0 | 4 | 181 |
| 365 | 10 | 29.7 | 17.2 | 51.2 | 4 | 181 |

TABLE 15
T03 Anti-Asia Type 1 antigen SN Titers Geometric Means-Positive
Control Vaccine

| | | | lower | upper | | |
|---|---|---|---|---|---|---|
| | | | 95% | 95% | | |
| | | | conf. | conf. | | |
| day of | no. | geometric | limit | limit | | |
| study | animals | mean | for mean | for mean | minimum | maximum |
| 14 | 10 | 152.0 | 98.3 | 235.0 | 45 | 362 |
| 29 | 10 | 97.0 | 52.8 | 178.2 | 23 | 362 |
| 60 | 10 | 75.9 | 43.7 | 131.8 | 32 | 256 |
| 90 | 10 | 78.7 | 45.0 | 137.6 | 16 | 181 |
| 120 | 10 | 90.5 | 47.8 | 171.0 | 23 | 362 |
| 149 | 10 | 97.0 | 51.2 | 183.8 | 23 | 256 |
| 180 | 10 | 123.4 | 70.2 | 217.0 | 45 | 256 |
| 210 | 10 | 111.2 | 63.5 | 194.5 | 45 | 256 |
| 240 | 10 | 127.8 | 79.8 | 204.6 | 45 | 256 |
| 270 | 10 | 90.6 | 52.2 | 157.2 | 23 | 256 |
| 300 | 10 | 103.7 | 61.5 | 175.0 | 45 | 256 |
| 330 | 10 | 87.2 | 49.9 | 152.6 | 32 | 256 |
| 365 | 10 | 90.3 | 52.4 | 155.5 | 45 | 256 |

TABLE 16
T04 Anti-Asia Type 1 antigen SN Titers Geometric Means-Positive
Control Vaccine

| | | | lower | upper | | |
|---|---|---|---|---|---|---|
| | | | 95% | 95% | | |
| | | | conf. | conf. | | |
| day of | no. | geometric | limit | limit | | |
| study | animals | mean | for mean | for mean | minimum | maximum |
| 14 | 10 | 187.2 | 121.0 | 289.5 | 45 | 362 |
| 29 | 10 | 115.3 | 62.8 | 211.8 | 32 | 362 |
| 60 | 10 | 103.8 | 59.8 | 180.3 | 16 | 256 |
| 90 | 10 | 115.2 | 65.8 | 201.4 | 32 | 256 |
| 120 | 10 | 107.5 | 56.8 | 203.1 | 32 | 256 |
| 149 | 10 | 115.3 | 60.8 | 218.5 | 32 | 256 |
| 180 | 10 | 141.9 | 80.7 | 249.6 | 64 | 256 |
| 210 | 10 | 137.0 | 78.3 | 239.8 | 64 | 256 |
| 240 | 10 | 141.9 | 88.6 | 227.2 | 64 | 256 |
| 270 | 10 | 119.2 | 68.7 | 206.9 | 45 | 256 |
| 300 | 10 | 132.2 | 78.4 | 223.0 | 64 | 256 |
| 330 | 10 | 141.8 | 81.0 | 248.1 | 64 | 256 |
| 365 | 10 | 115.2 | 66.8 | 198.5 | 64 | 256 |

TABLE 17
Statistical Analysis of Asia Type 1 antigen Responses

| | | least | | lower 95% | upper 95% | |
|---|---|---|---|---|---|---|
| | | squares | | conf. limit | conf. limit | |
| treatment | day of | mean | standard | for mean | for mean | |
| comparison | study | diff. | error | difference | difference | P-value |
| T02-T03 | 14 | −0.103 | 0.300 | −0.720 | 0.513 | 0.7334 |
| T02-T04 | 14 | −0.312 | 0.300 | −0.928 | 0.305 | 0.3085 |
| T03-T04 | 14 | −0.208 | 0.300 | −0.825 | 0.408 | 0.4937 |
| T02-T03 | 29 | −0.660 | 0.419 | −1.520 | 0.200 | 0.1268 |
| T02-T04 | 29 | −0.833 | 0.419 | −1.693 | 0.027 | 0.0571 |
| T03-T04 | 29 | −0.173 | 0.419 | −1.033 | 0.687 | 0.6833 |
| T02-T03 | 60 | −1.140 | 0.380 | −1.920 | −0.360 | 0.0058 |
| T02-T04 | 60 | −1.453 | 0.380 | −2.234 | −0.673 | 0.0007 |
| T03-T04 | 60 | −0.314 | 0.380 | −1.094 | 0.467 | 0.4168 |
| T02-T03 | 90 | −0.760 | 0.385 | −1.551 | 0.030 | 0.0588 |
| T02-T04 | 90 | −1.141 | 0.385 | −1.932 | −0.351 | 0.0063 |
| T03-T04 | 90 | −0.381 | 0.385 | −1.172 | 0.409 | 0.3312 |
| T02-T03 | 120 | −1.247 | 0.439 | −2.148 | −0.346 | 0.0085 |
| T02-T04 | 120 | −1.419 | 0.439 | −2.320 | −0.519 | 0.0032 |
| T03-T04 | 120 | −0.172 | 0.439 | −1.073 | 0.728 | 0.6979 |
| T02-T03 | 149 | −1.250 | 0.441 | −2.154 | −0.346 | 0.0085 |
| T02-T04 | 149 | −1.423 | 0.441 | −2.327 | −0.519 | 0.0032 |
| T03-T04 | 149 | −0.173 | 0.441 | −1.077 | 0.731 | 0.6980 |
| T02-T03 | 180 | −1.005 | 0.389 | −1.803 | −0.207 | 0.0155 |
| T02-T04 | 180 | −1.145 | 0.389 | −1.943 | −0.347 | 0.0066 |
| T03-T04 | 180 | −0.140 | 0.389 | −0.938 | 0.658 | 0.7221 |
| T02-T03 | 210 | −0.976 | 0.386 | −1.767 | −0.185 | 0.0175 |
| T02-T04 | 210 | −1.185 | 0.386 | −1.976 | −0.394 | 0.0048 |
| T03-T04 | 210 | −0.209 | 0.386 | −1.000 | 0.582 | 0.5921 |
| T02-T03 | 240 | −1.078 | 0.324 | −1.744 | −0.413 | 0.0026 |
| T02-T04 | 240 | −1.183 | 0.324 | −1.849 | −0.517 | 0.0011 |
| T03-T04 | 240 | −0.105 | 0.324 | −0.770 | 0.561 | 0.7498 |
| T02-T03 | 270 | −1.355 | 0.380 | −2.135 | −0.575 | 0.0014 |
| T02-T04 | 270 | −1.630 | 0.380 | −2.410 | −0.850 | 0.0002 |
| T03-T04 | 270 | −0.275 | 0.380 | −1.055 | 0.505 | 0.4755 |
| T02-T03 | 300 | −1.073 | 0.360 | −1.812 | −0.333 | 0.0061 |
| T02-T04 | 300 | −1.315 | 0.360 | −2.055 | −0.576 | 0.0011 |
| T03-T04 | 300 | −0.243 | 0.360 | −0.982 | 0.497 | 0.5066 |
| T02-T03 | 330 | −1.077 | 0.386 | −1.868 | −0.286 | 0.0095 |
| T02-T04 | 330 | −1.563 | 0.386 | −2.354 | −0.772 | 0.0004 |
| T03-T04 | 330 | −0.486 | 0.386 | −1.277 | 0.305 | 0.2185 |
| T02-T03 | 365 | −1.111 | 0.375 | −1.881 | −0.341 | 0.0063 |
| T02-T04 | 365 | −1.355 | 0.375 | −2.125 | −0.585 | 0.0012 |
| T03-T04 | 365 | −0.244 | 0.375 | −1.014 | 0.526 | 0.5214 |

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1-34. (canceled)

35. A vaccine comprising an FMD antigen and an adjuvant comprising oil, a polycationic carrier, and an immunostimulatory oligonucleotide, wherein said vaccine is a water-in-oil (W/O) emulsion, for use in preventing foot lesions caused by FMD infection in a ruminant, wherein said vaccine is administered as a single prime dose and one or more booster doses, wherein the first of said one or more booster doses is administered about 7 to about 12 months after the single prime dose.

36. The vaccine according to claim 35, wherein each consequent booster dose is administered 7 to about 12 months after the previous booster dose.

37. The vaccine according to claim 35 wherein the cationic carrier is DEAE Dextran.

38. The vaccine according to claim 37, wherein the DEAE Dextran is present in an amount of 25-250 mg per dose and the immunostimulatory oligonucleotide is present in an amount selected from the group consisting of 25-250 μg and 50-150 μg per dose.

39. The vaccine according to claim 35 wherein the vaccine comprises at least 40% oil v/v_to at least 60% oil v/v.

40. The vaccine according to claim 35 wherein the oil is a light mineral oil.

41. The vaccine according to claim 35 wherein the antigen is from a FMD virus of serotype O, A, C, and/or Asia 1.

42. The vaccine according to claim 35 wherein the antigen is from a FMD virus of serotype SAT1, SAT2 and/or SAT3.

43. The vaccine according to claim 35 wherein the antigen is selected from the group consisting of O1 Manisa, O1 BFS or Campos, Asia 1 Shamir, A Iran '96, A22 Iraq, SAT2 Saudi Arabia, A10-61, A5, A12, A24/Cruzeiro, C3/Indaial, O1, C1-Santa Pau, C1-C5, A22/550/Azerbaijan/65, SAT1-SAT3, A, A/TNC/71/94, A/IND/2/68, A/IND/3/77, A/IND/5/68, A/IND/7/82, A/IND/16/82, A/IND/17/77, A/IND/17/82, A/IND/19/76, A/IND/20/82, A/IND/22/82, A/IND/25/81, A/IND/26/82, A/IND/54/79, A/IND/57/79, A/IND/73/79, A/IND/85/79, A/IND/86/79, A/APA/25/84, A/APN/41/84, A/APS/44/05, A/APS/50/05, A/APS/55/05, A/APS/66/05, A/APS/68/05, A/BIM/46/95, A/GUM/33/84, A/ORS/66/84, A/ORS/75/88, A/TNAn/60/947/Asia/1, A/IRN/05, Asia/IRN/05, O/HK/2001, 0/UKG/3952/2001, O/UKG/4141/2001, Asia 1/HNK/CHA/05, Asia I/XJ, HK/70, O/UKG/7039/2001, O/UKG/9161/2001, O/UKG/7299/2001, O/UKG/4014/2001, O/UKG/4998/2001, O/UKG/9443/2001, O/UKG/5470/2001, O/UKG/5681/2001, O/ES/2001, HKN/2002, O5India, O/BKF/2/92, K/37/84/A, KEN/1/76/A, GAM/51/98/A, A10/Holland, O/KEN/1/91, O/IND49/97, O/IND65/98, O/IND64/98, O/IND48/98, O/IND47/98, O/IND82/97, O/IND81/99, O/IND81/98, O/IND79/97, O/IND78/97, O/IND75/97, O/IND74/97, O/IND70/97, O/IND66/98, O/IND63/97, O/IND61/97, O/IND57/98, O/IND56/98, O/IND55/98, O/IND54/98, O/IND469/98, O/IND465/97, O/IND464/97, O/IND424/97, O/IND423/97, O/IND420/97, O/IND414/97, O/IND411/97, O/IND410/97, O/IND409/97, O/IND407/97, O/IND399/97, O/IND39/97, O/IND391/97, O/IND38/97, O/IND384/97, O/IND380/97, O/IND37/97, O/IND352/97, O/IND33/97, O/IND31/97, O/IND296/97, O/IND23/99, O/IND463/97, O/IND461/97, O/IND427/98, O/IND28/97, O/IND287/99, O/IND285/99, O/IND282/99, O/IND281/97, O/IND27/97, O/IND278/97, O/IND256/99, O/IND249/99, O/IND210/99, O/IND208/99, O/IND207/99, O/IND205/99, O/IND185/99, O/IND175/99, O/IND170/97, O/IND164/99, O/IND160/99, O/IND153/99, O/IND148/99, O/IND146/99, O/SKR/2000, A22/India/17/77, O/IND/R2/1975 and any combination thereof.

44. The vaccine according to any one of claim 35, wherein the antigen is an inactivated FMD virus.

45. The vaccine according to claim 43, wherein the inactivated FMD virus is a recombinant virus.

46. The vaccine according to claim 44 wherein the recombinant FMD virus lacks functional leader protein.

47. The vaccine according to claim 44 wherein the recombinant FMD virus further comprises a DIVA markers.

48. The vaccine according to claim 45 wherein the recombinant FMD virus further comprises one or more deletion sequences as negative markers.

49. The vaccine according to claim 45 wherein the recombinant FMD virus further comprises one or mutations in capsid coding sequences inducing thermostability.

50. The vaccine according claim 45, wherein the recombinant FMD virus expresses a capsid from a heterologous FMD strain.

51. The vaccine according to claim 50, wherein the heterologous FMD strain is a strain of serotype A, or serotype O, or serotype Asia, or serotype C, or serotype SAT1, or serotype SAT2 or serotype SAT 3.

52. The vaccine according to claim 50, wherein the heterologous strain is selected from the group consisting of O1 Manisa, O1 BFS or Campos, Asia 1 Shamir, A Iran '96, A22 Iraq, SAT2 Saudi Arabia, A10-61, A5, A12, A24/Cruzeiro, C3/Indaial, O1, C1-Santa Pau, C1-C5, A22/550/Azerbaijan/65, SAT1-SAT3, A, A/TNC/71/94, A/IND/2/68, A/IND/3/77, A/IND/5/68, A/IND/7/82, A/IND/16/82, A/IND/17/77, A/IND/17/82, A/IND/19/76, A/IND/20/82, A/IND/22/82, A/IND/25/81, A/IND/26/82, A/IND/54/79, A/IND/57/79, A/IND/73/79, A/IND/85/79, A/IND/86/79, A/APA/25/84, A/APN/41/84, A/APS/44/05, A/APS/50/05, A/APS/55/05, A/APS/66/05, A/APS/68/05, A/BIM/46/95, A/GUM/33/84, A/ORS/66/84, A/ORS/75/88, A/TNAn/60/947/Asia/1, A/IRN/05, Asia/IRN/05, O/HK/2001, 0/UKG/3952/2001, O/UKG/4141/2001, Asia 1/HNK/CHA/05, Asia I/XJ, HK/70, O/UKG/7039/2001, O/UKG/9161/2001, O/UKG/7299/2001, O/UKG/4014/2001, O/UKG/4998/2001, O/UKG/9443/2001, O/UKG/5470/2001, O/UKG/5681/2001, O/ES/2001, HKN/2002, O5India, O/BKF/2/92, K/37/84/A, KEN/1/76/A, GAM/51/98/A, A10/Holland, O/KEN/1/91, O/IND49/97, O/IND65/98, O/IND64/98, O/IND48/98, O/IND47/98, O/IND82/97, O/IND81/99, O/IND81/98, O/IND79/97, O/IND78/97, O/IND75/97, O/IND74/97, O/IND70/97, O/IND66/98, O/IND63/97, O/IND61/97, O/IND57/98, O/IND56/98, O/IND55/98, O/IND54/98, O/IND469/98, O/IND465/97, O/IND464/97, O/IND424/97, O/IND423/97, O/IND420/97, O/IND414/97, O/IND411/97, O/IND410/97, O/IND409/97, O/IND407/97, O/IND399/97, O/IND39/97, O/IND391/97, O/IND38/97, O/IND384/97, O/IND380/97, O/IND37/97, O/IND352/97, O/IND33/97, O/IND31/97, O/IND296/97, O/IND23/99, O/IND463/97, O/IND461/97, O/IND427/98, O/IND28/97, O/IND287/99, O/IND285/99, O/IND282/99, O/IND281/97, O/IND27/97, O/IND278/97, O/IND256/99, O/IND249/99, O/IND210/99, O/IND208/99, O/IND207/99, O/IND205/99, O/IND185/99, O/IND175/99, O/IND170/97, O/IND164/99, O/IND160/99, O/IND153/99, O/IND148/99, O/IND146/99, O/SKR/2000, A22/India/17/77, O/IND/R2/75 and any combination thereof.

53. The vaccine according to claim 43 wherein the FMD virus is present in an amount selected from the group consisting of at least 4 μg, 8 μg, and 10 μg per dose per strain.

54. The vaccine according to claim 35 wherein the ruminant is a bovine and the ruminant is FMD-negative.
